(19) United States
(12) Patent Application Publication
(10) Pub. No.: US 2022/0296365 A1
(43) Pub. Date: Sep. 22, 2022

(54) Epicardial Anchor With Radial Slot

(71) Applicant: Tendyne Holdings, Inc., St. Paul, MN (US)

(72) Inventors: Preston James Huddleston, Maplewood, MN (US); Rebecca Nelson, Starbuck, MN (US); William Peckels, Robbinsdale, MN (US)

(73) Assignee: Tendyne Holdings, Inc., St. Paul, MN (US)

(21) Appl. No.: 17/684,684

(22) Filed: Mar. 2, 2022

Related U.S. Application Data
(60) Provisional application No. 63/161,479, filed on Mar. 16, 2021.

(51) Int. Cl. A61F 2/24

(52) U.S. Cl. CPC A61F 2/2418; A61F 2220/0008

(57) ABSTRACT

An epicardial anchor device may include a base and a hub each defining a radial slot. The hub is received within, and is rotatable relative to, the base. The radial slot of the hub defines a tether passageway for receiving a tether coupled to a prosthetic heart valve. The epicardial anchor device may have an unlocked configuration in which the radial slot of the base aligns with the radial slot of the hub so that the tether may be slid laterally through the radial slot of the base, and a locked configuration in which the radial slot of the base is out of alignment with the radial slot of the hub so that the tether cannot be slid laterally away from the radial center of the hub. Rotation of the hub may transition the epicardial anchor device from the locked to the unlocked configuration.

### CROSS-REFERENCE TO RELATED APPLICATIONS

This application claims the benefit of the filing date of U.S. Provisional Patent Application No. 63/161,479, filed Mar. 16, 2021, the disclosure of which is hereby incorporated by reference herein.

### BACKGROUND OF THE DISCLOSURE

Embodiments are described herein that relate to devices and methods for anchoring a medical device such as a prosthetic heart valve replacement.

Some known devices for anchoring a medical device, such as, for example, a prosthetic heart valve (e.g. mitral valve) can include securing one or more tethers extending from the medical device to body tissue. For example, one or more tethers can extend from a prosthetic heart valve through an opening in the ventricular wall of the heart. Some known methods of anchoring or securing the tethers can include the use of staples or other fasteners that engage or pierce tissue near the puncture site. Such devices can have relatively large profiles and be difficult to easily deliver percutaneously to the desired anchoring site. Some known methods of securing a prosthetic heart valve can include suturing the tethers extending from the valve to body tissue, or tying the suture ends. Such devices and methods can be difficult to maneuver to secure the tether(s) with a desired tension,

Other known devices may include anchors that include a central aperture through which the tether is passed, with the tether being secured within the central aperture after being passed therethrough. In some circumstances, it may be desirable to disconnect the tether from the anchor in order to re-tension the tether, or in order to replace the original anchor with another anchor. In these circumstances, it may be desirable to have an anchor that facilitates easy and rapid removal of the anchor from the tether and/or securement of the anchor to the tether.

### BRIEF SUMMARY OF THE DISCLOSURE

According to one aspect of the disclosure, an epicardial anchor device includes a base and a hub. The base defines an outer radial slot, and the hub defines an inner radial slot. The hub received at least partially within the base. The hub is rotatable about a center longitudinal axis relative to the base. The hub defines, at least in part, a tether passageway for receiving therethrough a tether coupled to a prosthetic heart valve, the tether passageway being continuous with the inner radial slot. The epicardial anchor device has a locked configuration in which the inner radial slot is discontinuous with the outer radial slot, and an unlocked configuration in which the inner radial slot is continuous with the outer radial slot. The epicardial anchor device is transitionable from the locked configuration to the unlocked configuration via rotation of the hub about the center longitudinal axis relative to the base in a first rotational direction. The epicardial anchor device is transitionable from the unlocked configuration to the unlocked configuration via rotation of the hub about the center longitudinal axis relative to the base in a second rotational direction opposite the first rotational direction.

According to another aspect of the disclosure, a method of implanting a prosthetic heart valve system includes implanting a prosthetic heart valve into a native heart valve annulus of a patient so that a tether has a first end fixedly coupled to the prosthetic heart valve and a second free end. An intermediate portion of the tether extends through a wall of a heart of the patient while the prosthetic heart valve is in the native heart valve annulus. The method includes sliding an epicardial anchor device over the intermediate portion of the tether so that the intermediate portion of the tether passes through an outer radial slot in a base of the epicardial anchor device and through an inner radial slot in a hub of the epicardial anchor device, until the intermediate portion of the tether is received within a central tether passageway of the epicardial anchor device. The inner radial slot is continuous with the outer radial slot during the sliding. The epicardial anchor device is positioned in contact with the wall of the heart. The tether is tensioned to a desired tension level. While the tether is tensioned to the desired tension level, the hub is rotated about a center longitudinal axis relative to the base so that the inner radial slot is discontinuous with the outer radial slot.

According to another aspect of the disclosure, a method of adjusting a prosthetic heart valve system includes accessing an epicardial anchor device after a first prosthetic heart valve implantation procedure has been performed. The first prosthetic heart valve implantation had included implanting the prosthetic heart valve into a native valve annulus of a patient and coupling the epicardial anchor device to the prosthetic heart valve via a tether fixed to the prosthetic heart valve so that the epicardial anchor device is positioned on an epicardial surface of the heart. After accessing the epicardial anchor device, the tether is gripped. While the tether is gripped, the epicardial anchor device is transitioned from a locked condition to an unlocked condition by rotating a hub of the epicardial anchor device about a center longitudinal axis relative to a base of the epicardial anchor device so that an inner radial slot of the hub is continuous with an outer radial slot of the base. This transition may be performed manually, for example with a user's thumb. After transitioning the epicardial anchor device to the unlocked condition, the epicardial anchor device is slid away from the tether so that the tether passes from a central tether passageway of the hub through the inner and outer radial slots until the epicardial anchor device no longer receives the tether.

According to another aspect of the disclosure, an epicardial anchor device includes a base defining a radial slot, and a hub defining a radial slot. The hub may be received at least partially within the base, and may be rotatable about a center longitudinal axis of the hub relative to the base. The radial slot of the hub may define, at least in part, a tether passageway for receiving therethrough a tether coupled to a prosthetic heart valve. The epicardial anchor device may have an unlocked configuration in which the radial slot of the base aligns with the radial slot of the hub so that the tether may be slid laterally through the radial slot of the base and the radial slot of the hub to position the tether at a radial center of the hub. The epicardial anchor device may have a locked configuration in which the radial slot of the base is out of alignment with the radial slot of the hub so that the tether cannot be slid laterally away from the radial center of the hub. The epicardial anchor device may be transitioned from the locked configuration to the unlocked configuration via rotation of the hub about the center longitudinal axis of the hub relative to the base in a first rotational direction, and transitioned from the unlocked configuration to the locked configuration via rotation of the hub about the center longitudinal axis of the hub relative to the base in a second rotational direction opposite the first rotational direction.

According to another aspect of the disclosure, a method of implanting a prosthetic heart valve system may include implanting a prosthetic heart valve into a native heart valve annulus of a patient so that a tether has a first end fixedly coupled to the prosthetic heart valve and a second free end. An intermediate portion of the tether may extend through a wall of a heart of the patient while the prosthetic heart valve is in the native heart valve annulus. The method may also include sliding an epicardial anchor device over the intermediate portion of the tether so that the intermediate portion of the tether passes through a radial slot in a base of the epicardial anchor device and through a radial slot in a hub of the epicardial anchor device, until the intermediate portion of the tether is received within a central tether passageway of the epicardial anchor device. The radial slot of the base may be aligned with the radial slot of the hub and may be continuous with the outer radial slot during the sliding. The method may further include positioning the epicardial anchor device in contact with the wall of the heart, and tensioning the tether to a desired tension level. While the tether is tensioned to the desired tension level, the hub may be rotated about a center longitudinal axis relative to the base to pin the tether to the epicardial anchor device.

According to yet a further aspect of the disclosure, a method of implanting a prosthetic heart valve system includes implanting a prosthetic heart valve into a native heart valve annulus of a patient so that a tether has a first end fixedly coupled to the prosthetic heart valve and a second free end. An intermediate portion of the tether may extend through a wall of a heart of the patient while the prosthetic heart valve is in the native heart valve annulus. The method may also include sliding an epicardial anchor device over the intermediate portion of the tether so that the intermediate portion of the tether passes laterally through a radial slot of the epicardial anchor device, until the intermediate portion of the tether is received within a central tether passageway of the epicardial anchor device. The epicardial anchor device may be positioned in contact with the wall of the heart. The epicardial anchor device may be manually actuated to drive a locking pin through the central tether passageway and through the tether to lock the tether to the epicardial anchor device at a first tether tension. A tether tensioning tool may be engaged with the epicardial anchor device while the tether is locked to the epicardial anchor device at the first tether tension. The tether may be fixed to the tether tensioning tool while the tether tensioning tool is engaged with the epicardial anchor device. After fixing the tether to the tether tensioning tool, the epicardial anchor device may be actuated using the tether tensioning tool to drive the locking pin away from the central tether passageway so that the locking pin disengages the tether. While the locking pin is disengaged with the tether, the tether tensioning tool may be used to adjust the tether to a second tether tension different than the first tether tension. While the tether is at the second tether tension, the tether tensioning tool may be used to drive the locking pin through the central tether passageway and through the tether to lock the tether to the epicardial anchor device at the second tether tension.

### BRIEF DESCRIPTION OF THE DRAWINGS

FIG. 1 is a cross-sectional illustration of portion of a heart with a prosthetic mitral valve implanted therein and an epicardial anchor device anchoring the mitral valve in position.

FIG. 2 is a schematic illustration of an epicardial anchor device, according to an embodiment.

FIG. 3A is a top perspective view of an epicardial anchor device, according to another embodiment.

FIG. 3B is a top view of the epicardial anchor device of FIG. 3A.

FIG. 3C is an exploded view of the epicardial anchor device of FIG. 3A.

FIG. 3D is a cross-sectional perspective view of the epicardial anchor device of FIG. 3A with a locking pin of the device shown in a first position.

FIG. 3E is a cross-sectional side view of the epicardial anchor device of FIG. 3A with the locking pin of the device shown in the first position.

FIG. 3F is a cross-sectional bottom perspective view of the epicardial anchor device of FIG. 3A with the locking pin shown in a second position.

FIGS. 3G and 3H are a top perspective and a bottom perspective view, respectively, of a hub member of the epicardial anchor device of FIG. 3A.

FIG. 3I is an enlarged top view of a portion of the pericardial pad device of FIG. 3A.

FIG. 4 is a perspective view of the epicardial anchor device of FIG. 3A with a delivery device coupled thereto.

FIGS. 5A-B are top views of an epicardial anchor device according to another embodiment of the disclosure in an unlocked condition.

FIG. 5C is a perspective view of the epicardial anchor device of FIGS. 5A-B positioned adjacent a tether.

FIG. 5D is a perspective view of the epicardial anchor device of FIGS. 5A-B receiving the tether of FIG. 5C.

FIG. 5E is a perspective view of the epicardial anchor device and tether of FIG. 5D, the epicardial anchor device being in a locked condition.

FIG. 5F is a top view of the epicardial anchor device of FIGS. 5A-B in the locked condition.

FIGS. 6A-B are perspective solid and transparent views, respectively, of an epicardial anchor device in an unpinned condition according to another embodiment of the disclosure.

FIGS. 6C-D are transparent side views of the epicardial anchor device of FIGS. 6A-B in an unpinned and pinned condition, respectively.

FIG. 6E is a transparent perspective view of the epicardial anchor device of FIGS. 6A-D in the pinned condition.

FIG. 7A is a side view of an epicardial anchor device according to another embodiment of the disclosure, in an open and pinned condition.

FIG. 7B is a transparent side view of the epicardial anchor device of FIG. 7A in an open and unpinned condition.

FIG. 7C is a perspective transparent view of the epicardial anchor device of FIG. 7A in an open and unpinned condition.

FIG. 7D is a side view of the epicardial anchor device of FIG. 7A in a closed and pinned condition.

FIG. 7E is a transparent side view of the epicardial anchor device of FIG. 7A in a closed and pinned condition.

FIG. 7F is a transparent perspective view of the epicardial anchor device of FIG. 7A in a closed and pinned condition.

FIG. 8A is a front view of an epicardial anchor device according to another aspect of the disclosure.

FIG. 8B is an exploded perspective view of the epicardial anchor device of FIG. 8A.

FIG. 8C is a perspective view of the base of the epicardial anchor device of FIG. 8A.

FIGS. 8D and 8E are cross-sections of the base of FIG. 8C taken at different depths.

FIG. 8F is a perspective view of a locking pin of the epicardial anchor device of FIG. 8A.

FIG. 8G is a front view of an actuator of the epicardial anchor device of FIG. 8A.

FIGS. 8H-I are front and rear perspective views, respectively, of the actuator of FIG. 8G.

### DETAILED DESCRIPTION OF THE DISCLOSURE

As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof.

As used herein, the words “proximal” and “distal” refer to a direction closer to and away from, respectively, an operator of, for example, a medical device. Thus, for example, the end of the medical device closest to the patient's body (e.g., contacting the patient's body or disposed within the patient's body) would be the distal end of the medical device, while the end opposite the distal end and closest to, for example, the user (or hand of the user) of the medical device, would be the proximal end of the medical device.

In some embodiments, an epicardial pad system is described herein that can be used to anchor a compressible prosthetic heart valve replacement (e.g., a prosthetic mitral valve), which can be deployed into a closed beating heart using a transcatheter delivery system. Such an adjustable-tether and epicardial pad system can be deployed via a minimally invasive procedure such as, for example, a procedure utilizing the intercostal or subxyphoid space for valve introduction. In such a procedure, the prosthetic valve can be formed in such a manner that it can be compressed to fit within a delivery system and secondarily ejected from the delivery system into the target location, for example, the mitral or tricuspid valve annulus.

A compressible prosthetic mitral valve can have a shape, for example that features a tubular stent body that contains leaflets and an atrial cuff. This allows the valve to seat within the mitral annulus and be held by the native mitral leaflets. The use of a flexible valve attached using an apical tether can provide compliance with the motion and geometry of the heart. The geometry and motion of the heart are well-known as exhibiting a complicated biphasic left ventricular deformation with muscle thickening and a sequential twisting motion. The additional use of the apically secured ventricular tether helps maintain the prosthetic valve's annular position without allowing the valve to migrate, while providing enough tension between the cuff and the atrial trabeculations to reduce, and preferably eliminate, perivalvular leaking. The use of a compliant valve prosthesis and the special shape and features can help reduce or eliminate clotting and hemodynamic issues, including left ventricular outflow tract (LVOT) interference problems. Many known valves are not able to address problems with blood flow and aorta/aortic valve compression issues.

Structurally, the prosthetic heart valve can include: a self-expanding tubular frame having a cuff at one end (the atrial end); one or more attachment points to which one or more tethers can be attached, preferably at or near the ventricular end of the valve; and a leaflet assembly that contains the valve leaflets, which can be formed from stabilized tissue or other suitable biological or synthetic material. In one embodiment, the leaflet assembly may include a wire form where a formed wire structure is used in conjunction with stabilized tissue to create a leaflet support structure, which can have anywhere from 1, 2, 3 or 4 leaflets, or valve cusps disposed therein. In another embodiment, the leaflet assembly can be wireless and use only the stabilized tissue and stent body to provide the leaflet support structure, and which can also have anywhere from 1, 2, 3 or 4 leaflets, or valve cusps disposed therein.

The upper cuff portion may be formed by heat-forming a portion of a tubular nitinol structure (formed from, for example, braided wire or a laser-cut tube) such that the lower portion retains the tubular shape but the upper portion is opened out of the tubular shape and expanded to create a widened collar structure that may be shaped in a variety of functional regular or irregular funnel-like or collar-like shapes.

A prosthetic mitral valve can be anchored to the heart at a location external to the heart via one or more tethers coupled to an anchor device, as described herein. For example, the tether(s) can be coupled to the prosthetic mitral valve and extend out of the heart and be secured at an exterior location (e.g., the epicardial surface) with an anchor device, as described herein. An anchor device as described herein can be used with one or more such tethers in other surgical situations where such a tether may be desired to extend from an intraluminal cavity to an external anchoring site.

The prosthetic heart valve may take other forms, for example an outer stent that is coupled to an inner stent, with the inner stent housing the prosthetic leaflets, and the tether attached to a ventricular end of the inner stent. Such configurations are described in greater detail in U.S. Patent Application Publication No. 2017/0196688, the disclosure of which is hereby incorporated by reference herein. Various examples of epicardial pads and methods of using the same are described in more detail in U.S. Patent Application Publication No. 2016/0143736, the disclosure of which is hereby incorporated by reference herein.

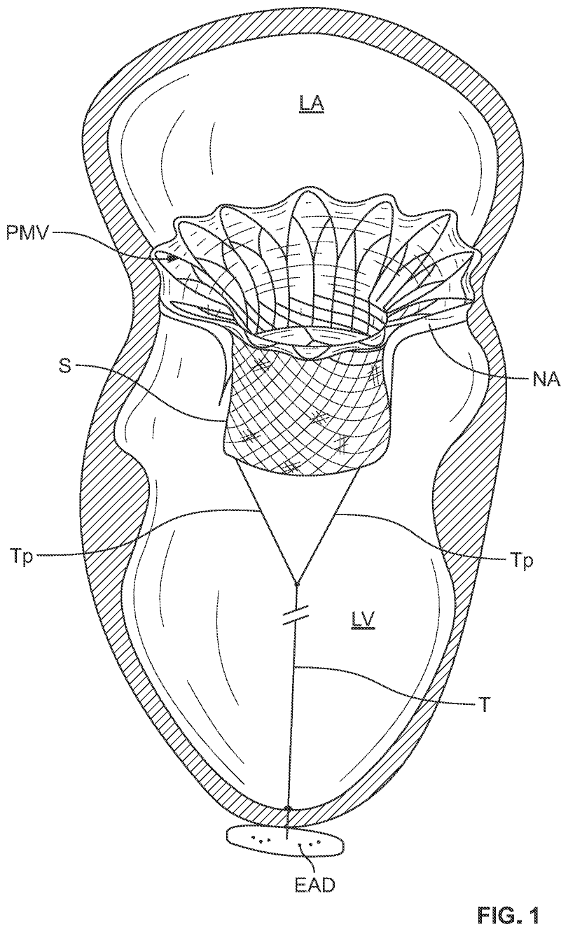

FIG. 1 is a cross-sectional illustration of the left ventricle LV and left atrium LA of a heart having a transcatheter prosthetic mitral valve PMV deployed therein and an epicardial anchor device EAD as described herein securing the prosthetic mitral valve PMV in place. FIG. 1 illustrates the prosthetic mitral valve PMV, including a stent S, seated into the native valve annulus NA and held there using an atrial cuff of the prosthetic mitral valve PMV, the radial tension from the native leaflets, and a ventricular tether T secured with attachment portions Tp to the prosthetic mitral valve PMV and to the epicardial anchor EAD. Various embodiments of an epicardial anchor device are described in more detail below with reference to specific embodiments.

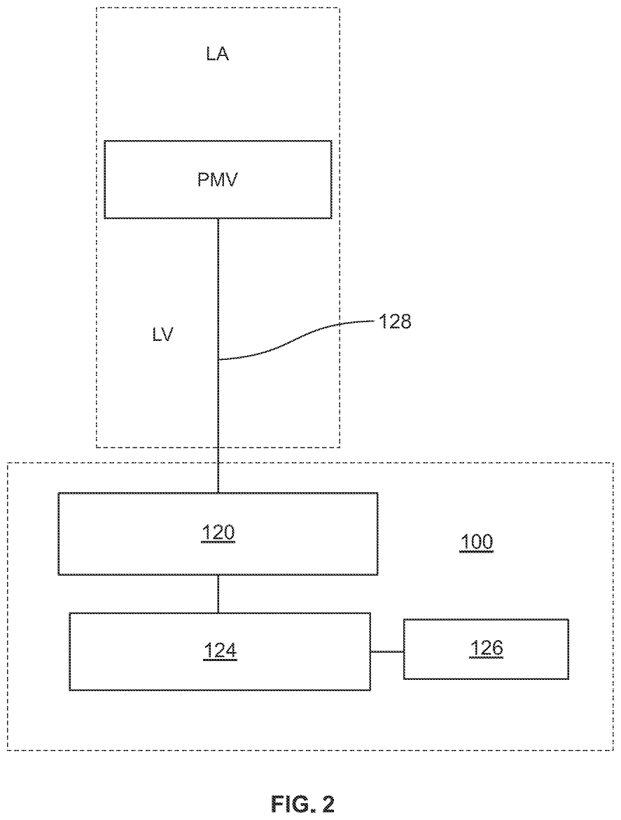

FIG. 2 is a schematic illustration of an epicardial anchor device 100 (also referred to herein as “anchor device” or “epicardial anchor”) according to an embodiment. The anchor device 100 can be used to anchor or secure a prosthetic mitral valve PMV deployed between the left atrium and left ventricle of a heart. The anchor device 100 can be used, for example, to anchor or secure the prosthetic mitral valve PMV via a tether 128 as described above with respect to FIG. 1. The anchor device 100 can also seal a puncture formed in the ventricular wall (not shown in FIG. 2) of the heart during implantation of the prosthetic mitral valve PMV. The anchor device 100 can also be used in other applications to anchor a medical device (such as any prosthetic atrioventricular valve or other heart valve) and/or to seal an opening such as a puncture.

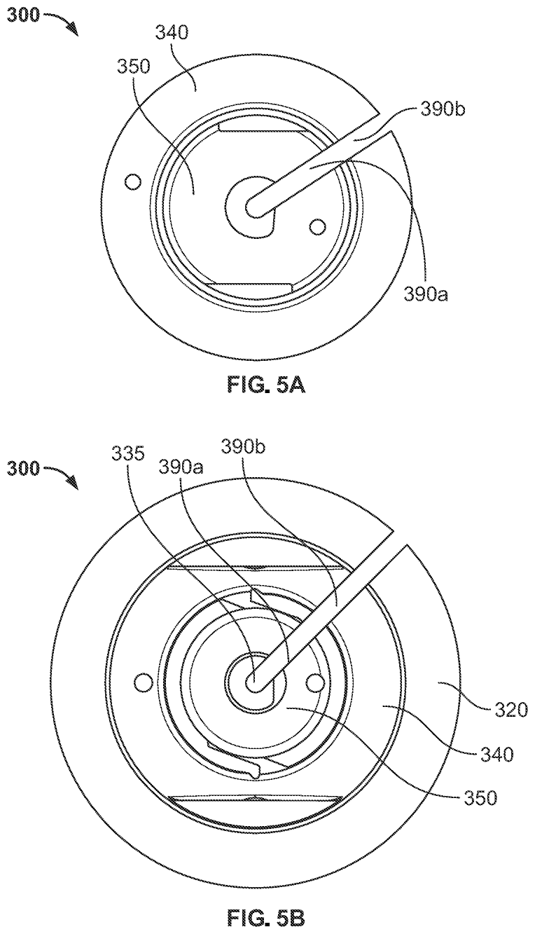

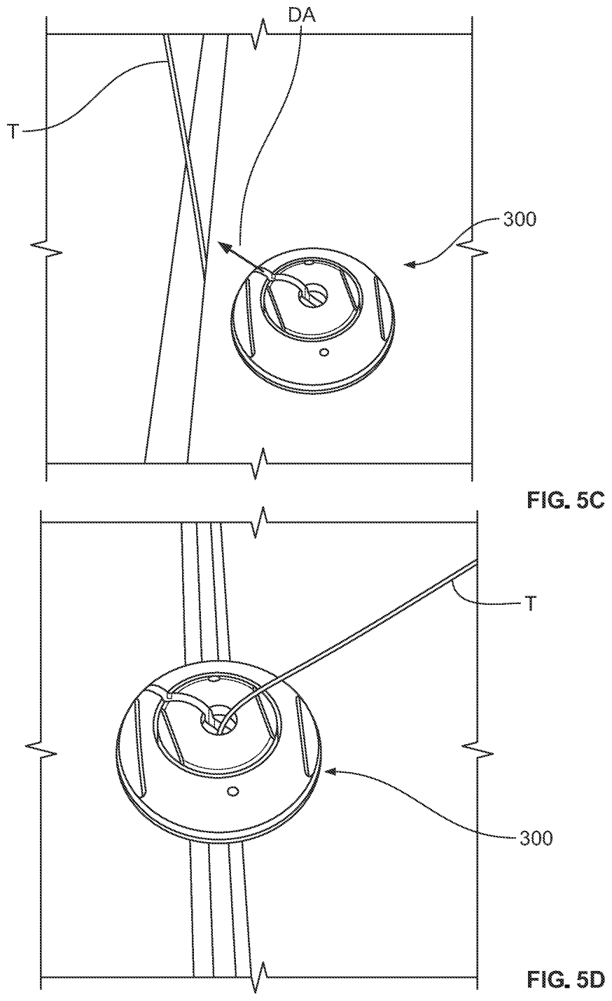

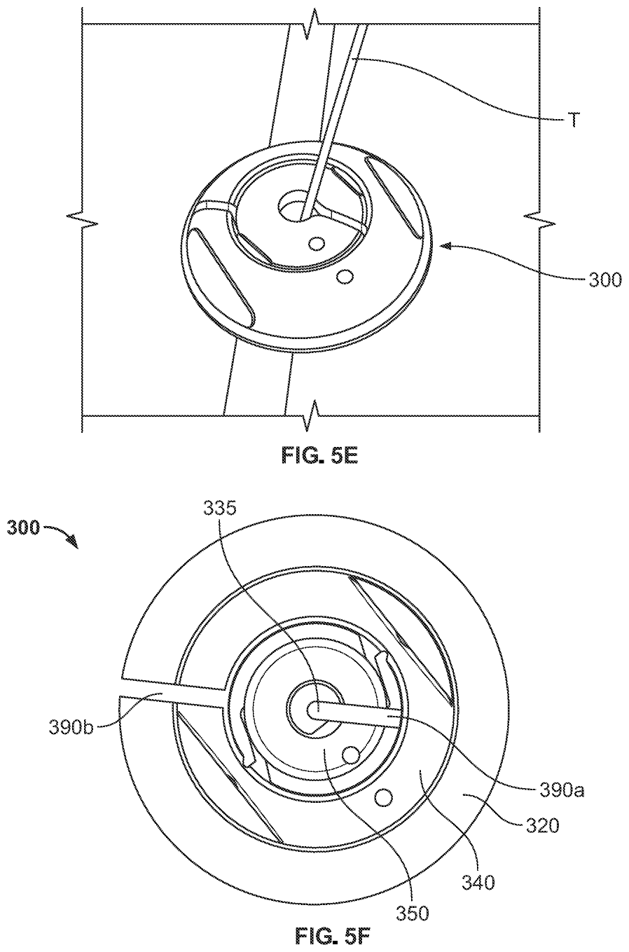

The anchor device 100 can include a pad (or pad assembly) 120, a tether attachment member 124 and a locking pin 126. The pad 120 can contact the epicardial surface of the heart and can be constructed of any suitable biocompatible surgical material. The pad 120 can be used to assist the sealing of a surgical puncture (e.g. a transapical puncture at or near the apex of the left ventricle) formed when implanting a prosthetic mitral valve. In some embodiments, the pad 120 can include a slot that extends radially to an edge of the pad 120 such that the pad 120 can be attached to, or disposed about, the tether 128 by sliding the pad 120 onto the tether 128 via the slot. Such an embodiment is described below with respect to FIGS. 5A-F.

In some embodiments, the pad 120 can be made with a double velour material to promote ingrowth of the pad 120 into the puncture site area. For example, pad or felt pledgets can be made of a felted polyester and may be cut to any suitable size or shape, such as those available from Bard® as PTFE Felt Pledgets having a nominal thickness of between 2.5 mm and 3.0 mm, including for example 2.6 mm, 2.7 mm, 2.8 mm, or 2.9 mm. In some embodiments, the pad 120 can be larger in diameter than the tether attachment member 124. The pad 120 can have a circular or disk shape, or other suitable shapes.

The tether attachment member 124 can provide the anchoring and mounting platform to which one or more tethers 128 can be coupled (e.g., tied or pinned). The tether attachment member 124 can include a base member (not shown) that defines at least a portion of a tether passageway (not shown) through which the tether 128 can be received and pass through the tether attachment member 124, and a locking pin channel (not shown) through which the locking pin 126 can be received. The locking pin channel can be in fluid communication with the tether passageway such that when the locking pin 126 is disposed in the locking pin channel, the locking pin 126 can contact or pierce the tether 128 as it passes through the tether passageway as described in more detail below with reference to specific embodiments.

The locking pin 126 can be used to hold the tether 128 in place after the anchor device 100 has been tightened against the ventricular wall and the tether 128 has been pulled to a desired tension. For example, the tether 128 can extend through a hole in the pad 120, and through the tether passageway of the tether attachment member 124. The locking pin 126 can be inserted or moved within a locking pin channel such that it pierces or otherwise engages the tether 128 as the tether 128 extends through the tether passageway of the tether attachment member 124. Thus, the locking pin 126 can intersect the tether 128 and secure the tether 128 to the tether attachment member 124.

The tether attachment member 124 can be formed with, a variety of suitable biocompatible material. For example, in some embodiments, the tether attachment member 124 can be made of polyethylene, or other hard or semi-hard polymer, and can be covered with a polyester velour to promote ingrowth. In other embodiments, the tether attachment member 124 can be made of metal, such as, for example, Nitinol®, or ceramic materials. The tether attachment member 124 can be various sizes and/or shapes. For example, the tether attachment member 124 can be substantially disk shaped.

In some embodiments, the tether attachment member 124 can include a hub that is movably coupled to the base member of tether attachment member 124. The hub can define a channel that can receive a portion of the locking pin (or locking pin assembly) 126 such that as the hub is rotated, the hub acts as a cam to move the locking pin 126 linearly within the locking pin channel. As with previous embodiments, as the locking pin 126 is moved within the locking pin channel, the locking pin can engage or pierce the tether 128 disposed within the tether passageway and secure the tether 128 to the tether attachment member 124.

In use, after a PMV has been placed within a heart, the tether extending from the PMV can be inserted into the tether passageway of the anchor device 100 and the tension on the tether attachment device can be adjusted to a desired tension. The anchor device 100 (e.g., some portion of the anchor device such as the tether attachment member 124, or the hub) can be actuated such that the locking pin 126 intersects the tether passageway and engages a portion of the tether disposed within the tether passageway, securing the tether to the tether attachment member. In some embodiments, prior to inserting the tether into the tether passageway, the anchor device 100 can be actuated to configure the anchor device 100 to receive the tether. In some embodiments, the anchor device 100 can be actuated by rotating a hub relative to a base member of the tether attachment member 124 such that the locking pin 126 is moved from a first position in which the locking pin is spaced from the tether passageway and a second position in which the locking pin intersects the tether passageway and engages or pierces the portion of the tether.

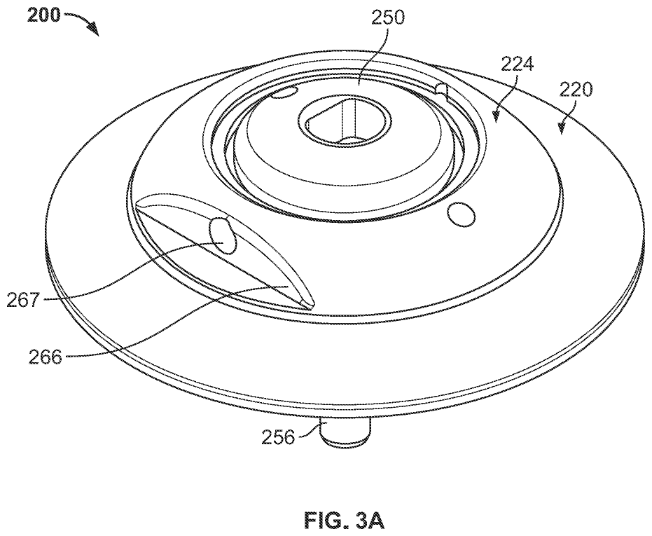

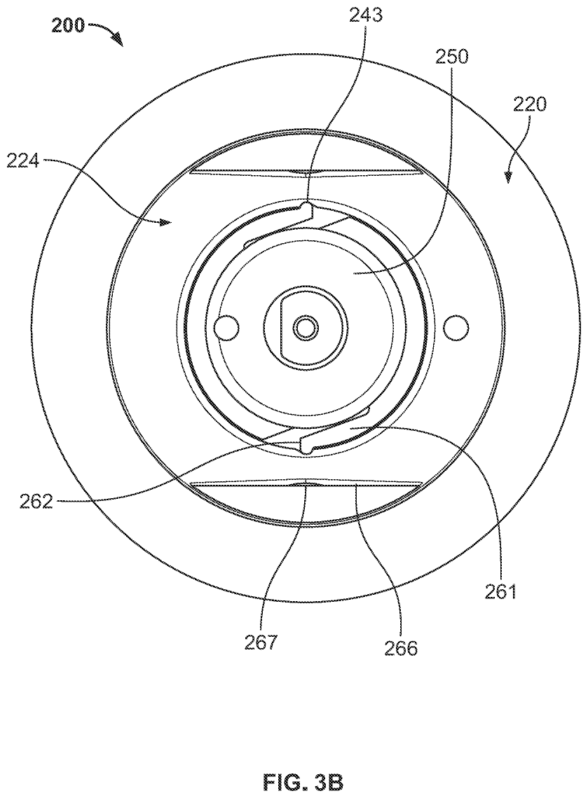

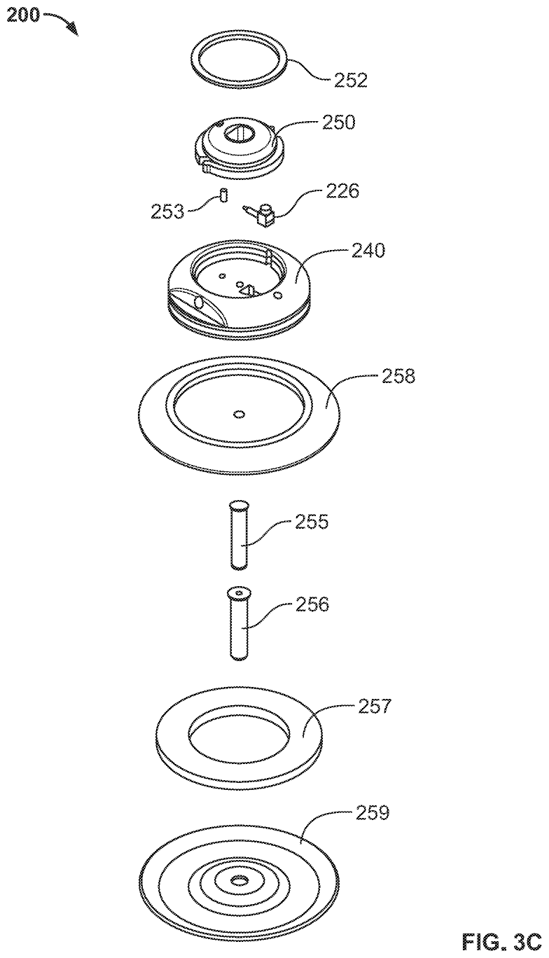

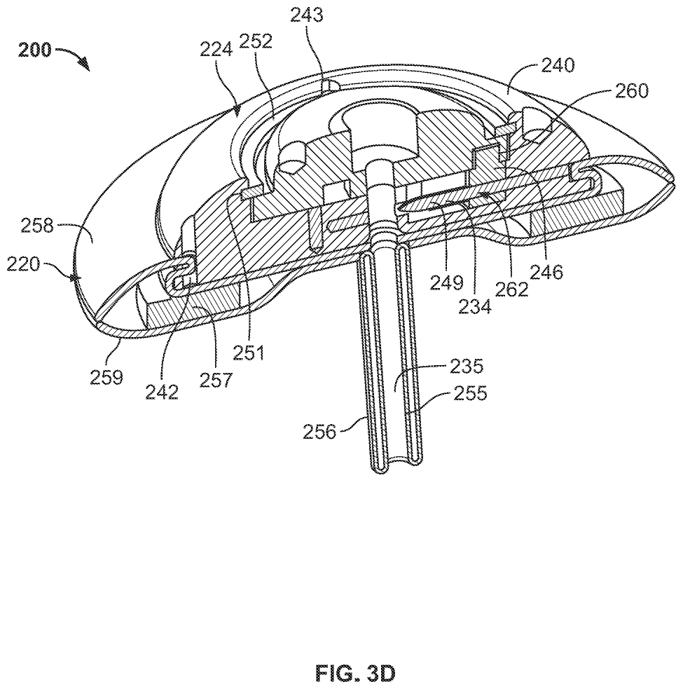

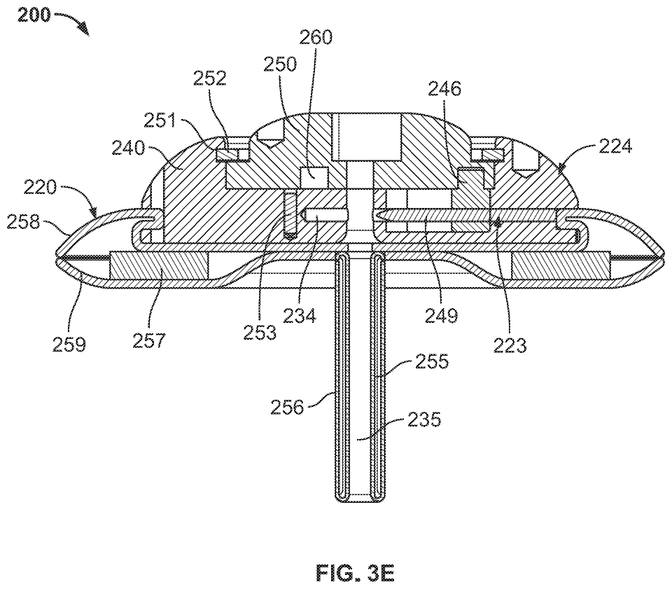

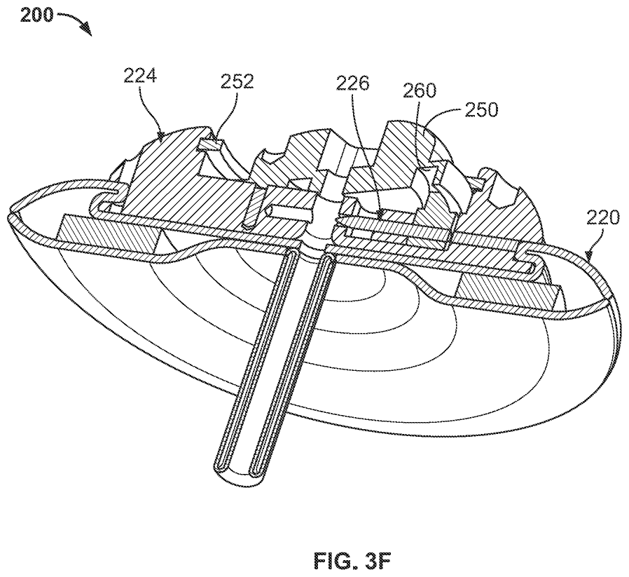

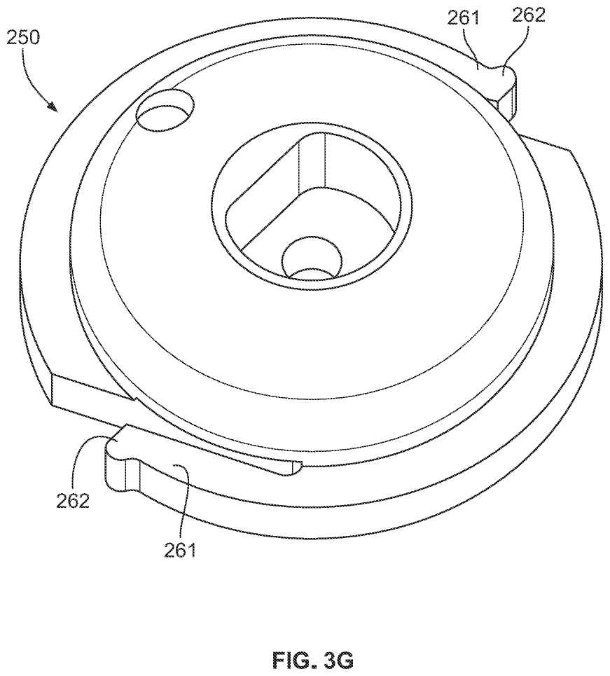

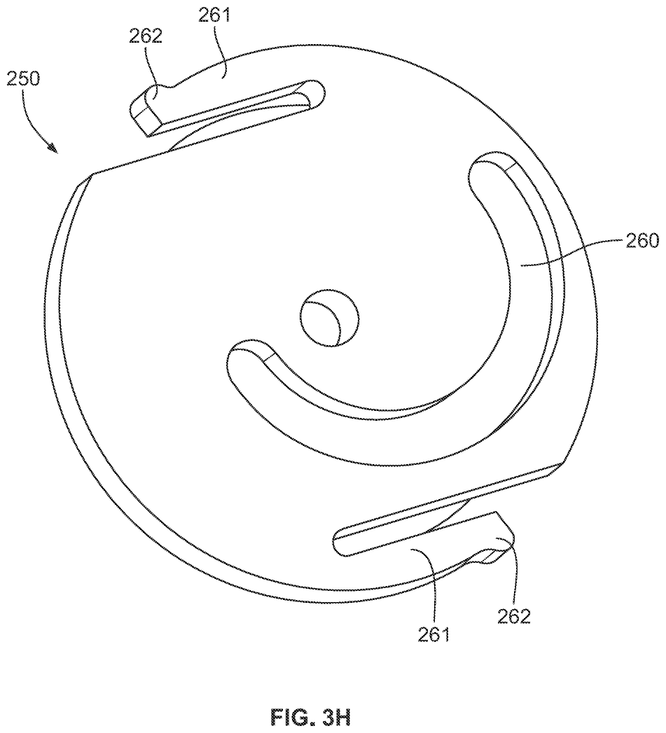

FIGS. 3A-I illustrate an epicardial anchor device according to another embodiment. An epicardial anchor device 200 includes a tether attachment member 224, a pad assembly 220, a tube member 255 and a tube cover member 256. The tether attachment member 224 includes a base member 240, a hub 250, a retaining ring 252, a locking pin assembly 226, and a pin member 253. The locking pin assembly 226 includes a driver portion 246 and a piercing portion 249. The base member 240 defines a circumferential pad channel 242, a retaining channel 251 and a locking pin channel 234. The pad channel 242 can be used to couple the pad assembly 220 to the tether attachment member 224. The retaining channel 251 can receive an outer edge of the retaining ring 252, which is used to retain the hub 250 to the base member 240. The base member 240 also defines cutouts or detents 243, as shown for example, in FIGS. 3B, 3D and 3I.

The tube member 255 is coupled to the base member 240 and the base member 240, the hub 250 and the tube member 255 collectively define a tether passageway 235 through which a tether (not shown) can be received. The cover member 256 can be formed with a fabric material, such as for example, Dacron®. The tether channel 235 intersects the locking pin channel 234 and is in fluid communication therewith.

The pad assembly 220 includes a top pad portion 258, a bottom pad portion 259 and a filler member 257 disposed therebetween. The top pad portion 258 and the bottom pad portion 259 can each be formed with, for example, a flexible fabric material. The top pad portion 258 and the bottom pad portion 259 can each define a central opening through which the tube member 255 can pass through. A portion of the top pad portion 258 is received within the channel 242 of the base member 240 as shown, for example, in FIGS. 3D-F.

An outer perimeter portion of the hub 250 is received within the retaining channel 251 such that the hub 250 can rotate relative to the base member 240 to actuate the locking pin assembly 226 as described in more detail below. As shown, for example, in FIGS. 3G and 3H, the hub 250 includes arms 261 with protrusions 262, which may also be referred to as detents or detent protrusions. The protrusions 262 can be received within cutouts 243 of the base member 240 and act as a stop or limit to the rotation of the hub 250. The slots 263 defined by the hub 250 enable the arms 261 to flex and allow the protrusions 262 to be moved in and out of the cutouts 243. As shown, for example, in FIGS. 3F and 3H the hub 250 defines a curved channel 260 on a bottom portion of the hub 250. The curved channel 260 is asymmetrical (or spiral) and receives the driver portion 246 of the locking pin assembly 226. As the hub 250 is rotated relative to the base member 240, the hub 250 acts as a cam to move the locking pin assembly 226 linearly within the locking pin channel 234. The locking pin assembly 226 can be moved from a first position in which the piercing portion 249 is disposed outside of the tether passageway 235 as shown in FIGS. 3D and 3E, and a second position in which the piercing portion 249 extends through the tether passageway 235 as shown in FIG. 3F. The pin member 253 (see, e.g., FIG. 3E) can be formed with a metal material that is more radio-opaque than the other components of the anchor device and thus visible to the user (e.g. physician) using conventional imaging modalities to enable the user to confirm that the locking pin assembly 226 has been fully moved to the second position.

In use, when the locking pin assembly 226 is in the first position, a tether (not shown) coupled to, for example, a prosthetic mitral valve and extending through a puncture site in the ventricular wall of a heart can be inserted through the tether passageway 235. The hub 250 can then be rotated 180 degrees to move the locking pin assembly 226 linearly within the locking pin channel 234 such that the piercing portion 249 extends through the tether passageway 235 and engages or pierces the tether, securing the tether to the tether attachment member 224. For example, when the locking pin is in the first position, the protrusions 262 of the hub 250 are each disposed within one of the cutouts 243 of the base member 240 (i.e., a first protrusion is in a first cutout, and a second protrusion is in a second cutout). The hub 250 can then be rotated 180 degrees such that the protrusions 262 are moved out of the cutouts 243 of the base member 240 and at the end of the 180 degrees the protrusions 262 are moved into the other of the cutouts 243 of the base member 240 (i.e., the first protrusion is now in the second cutout, the second protrusion is now in the first cutout).

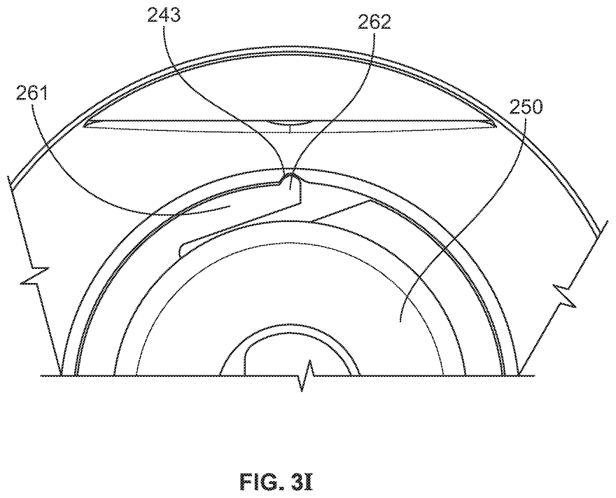

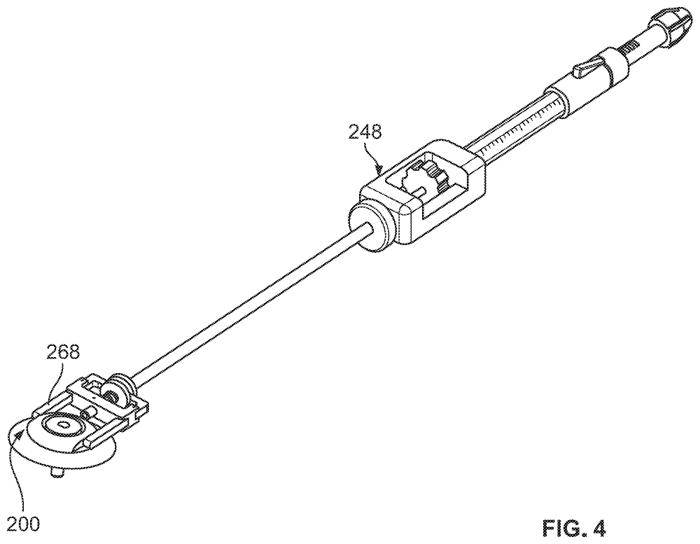

The base member 240 can also include cutout sections 266 and define side openings 267 (see, e.g., FIGS. 3A and 3B) that can be used to couple a delivery device to the epicardial anchor device 200. For example, FIG. 4 illustrates a delivery device 248 having coupling arms 268 and coupling pins (not shown) extending inwardly from the arms 268. The side openings 267 can receive the coupling pins and the cutout sections 266 can be engaged by the coupling arms 268.

In a typical use of epicardial anchor device 200 during a prosthetic mitral valve replacement, the prosthetic mitral valve is positioned within the patient's native mitral valve annulus through a transapical puncture, and a tether that is fixed to the prosthetic mitral valve extends through the transapical puncture so that it can be manipulated by a surgeon. The tether is passed through the tether passageway 235 and the epicardial anchor pad 200 is advanced over the tether into contact with the patient's heart. While the epicardial anchor pad 200 is in contact with the patient's heart, the tether is tensioned to a desired amount, for example using the delivery device 248, and then the tether is fixed at that desired tension using the pinning mechanism described in connection with epicardial anchor device 200. After the pinning is completed, and the surgeon is satisfied with the result of the implantation, the remaining length of the tether extending beyond the epicardial anchor device 200 is cut or otherwise trimmed to remove any excessive length of tether that would otherwise remain in the patient's body. When the excess length of tether is cut, it may be desirable to still leave about 5 cm of tether length to allow for future manipulation of the tether, if necessary. However, other lengths, such as about 1, 2, 3, 4, 5, 6, 7, 8, 9, or 10 cm, may be suitable. In some circumstances, after the excess length of tether is cut, it may become necessary or desirable to further manipulate the epicardial anchor device 200 and/or the tether for various reasons, either immediately after cutting the excess length of the tether, or after days, weeks, months, or more have passed after the implantation procedure is completed. As noted above the tether is locked to the epicardial anchor device 200 at a desired tension during the initial implantation. In some circumstances, as time passes, the tension on the tether may decrease compared to the initially set tension. This may occur, for example, due to the anatomy changing, for example via ventricular remodeling (or shrinking) or otherwise acclimating to prosthetic mitral valve implant assembly. In some instances, the initial tension placed on the tether may be too small. Still further, it may be desirable to replace the first epicardial pad with another epicardial pad. In some circumstances, the ventricular tissue in contact with the epicardial pad may begin to dimple as a result of the force applied against the ventricular tissue by the epicardial pad. If the ventricle begins to dimple, and the epicardial pad sits within the dimpled area, the tension on the tether may decrease from the initially set tether tension. In this situation, it may be desirable to replace the first epicardial pad with a second epicardial pad that has a larger surface area of contact with the patient's heart compared to the first epicardial pad. By switching to an epicardial anchor pad with a relatively large surface area of contact with the patient's heart wall, the pressure on the heart tissue from the epicardial pad may be reduced, because the same tension force is applied over a larger surface area. The larger surface area may also prevent the larger epicardial pad from sitting within any dimpled area of the ventricular tissue. A reduction in pressure on the heart wall tissue may also reduce the likelihood of damage (or severity of damage) that may be caused on the heart wall tissue as a result of the contact force between the epicardial anchor device and the heart wall tissue. In other words, if dimpling occurred with a smaller epicardial pad, it may be less likely for dimpling to occur again after a smaller epicardial pad is switched out with a larger epicardial pad.

It may be difficult to switch out a first epicardial pad with a second epicardial pad, for example because it may be difficult to slide the original epicardial anchor device off the tether, and it may also be difficult to slide the new epicardial pad over the tether. For example, epicardial anchor device 200 has a central tether passageway 235 that would require the epicardial anchor device 200 to be slid proximally along the remaining length of the gripped tether to remove the epicardial anchor device, which may be practically difficult and/or time consuming And if another different epicardial anchor device (e.g. one with a larger surface area of contact) is to replace the original pad, it may again be difficult to perform this replacement if the epicardial anchor device must be slid over the remaining length of tether being gripped outside of the patient's heart. Some of these challenges may be mitigated or eliminated by introducing certain design feature changes to epicardial anchor device 200, as described below.

FIGS. 5A-B are top views of an epicardial anchor device 300 according to another embodiment of the disclosure. Epicardial anchor device 300 is identical to epicardial anchor device 200 in all respects, other than the differences described below. Thus, the description of the features of epicardial anchor device 200 that are also found in epicardial anchor device 300 are not described here again, but it should be understood that, unless otherwise noted, the description and figures relating to epicardial anchor device 200 apply with equal force to epicardial anchor device 300. As is described in greater detail below, one difference between epicardial anchor device 200 and 300 is that epicardial anchor device 300 includes a radial slot extending from the central tether passageway through an outer circumferential rim of the epicardial anchor device 300 (when the epicardial anchor device 300 is in an unlocked condition) to allow for the tether to be slid into the epicardial anchor device 300 from the side, eliminating the requirement that the epicardial anchor device is threaded over a free end of the tether, as in epicardial anchor device 200.

FIGS. 5A-B both illustrate the hub 350 and the base 340 of the epicardial anchor device 300 while the epicardial anchor device 300 is in an unlocked condition. The pad assembly 320 is illustrated in FIG. 5B but omitted from FIG. 5A. The epicardial anchor device 300 includes a radial slot extending from the longitudinal center of the epicardial anchor device through an outer circumferential rim of the epicardial anchor device so that the center of the tether passageway 335 is accessible via the outer circumferential rim of the epicardial anchor device when in the unlocked condition. It should be understood that the epicardial anchor device 300 may include all of the components of epicardial anchor device 200, with the radial slot of the epicardial anchor device 300 avoiding interference with the pinning mechanism described in connection with epicardial anchor device 200. For example, referring to FIG. 3H, the radial slot may extend from the central aperture of the hub 350 so that the slot does not pass through the curved channel (labelled 260 for hub 250 in FIG. 3H) in which the locking pin assembly is received. Another difference may be that in epicardial anchor device 300, the retaining ring (labelled 252 in FIG. 3C) may be affixed to base 340 (via adhesive or another mechanism) to help prevent the retaining ring from rotating as the hub 350 is rotated. These features are described in greater detail below.

Referring back to FIGS. 5A-B, the radial slot in epicardial anchor device 300 may be thought of as including at least two separate portions, including an inner radial slot 390a and an outer radial slot 390b. The inner radial slot 390a may be formed in the hub 350, and the outer radial slot 390b may be formed in the base 340 and the pad assembly 320. When the epicardial anchor device 300 is in the unlocked condition (where the piercing portion of the locking pin assembly does not traverse the tether passageway, as shown in FIG. 3E), the inner and outer radial slots 390a, 390b may be continuous with each other.

FIG. 5C shows the epicardial anchor device 300 in the unlocked condition positioned adjacent a tether T of a prosthetic heart valve PMV. FIG. 5C illustrates the tether T passing through a gap, with the gap representing a transapical puncture, and the surfaces adjacent the gap representing the epicardial surface of the heart in which the transapical puncture is formed. In operation, with the user gripping the tether T (manually and/or with the help of a tether gripping device), the epicardial anchor device 300 may be slid in a direction toward the tether T represented by directional arrow DA, so that the tether T passes into the radial slot. As shown in FIG. 5D, the epicardial anchor device 300 may be slid over tether T until the tether T is positioned within the tether passageway 335 at the center of the epicardial anchor device 300. As described in connection with epicardial anchor device 200, the tether T may be tensioned to the desire level while the tether T is received within the tether passageway 335, at which point the hub 350 may be rotated (for example about 180 degrees) relative to the base 340. This relative rotation drives the locking pin assembly through the tether passageway 335 to pierce the tether T in the same manner as described above in connection with epicardial anchor device 200, thus locking the tether T at the desired level of tension.

FIGS. 5E-F illustrate the epicardial anchor device 300 after the hub 350 has been rotated relative to the base 340 and pad assembly 320 into the locked condition to pin the tether T at the desired tension. In the locked condition, the inner radial slot 390a is no longer continuous with the outer radial slot 390b. As should be understood from FIGS. 5A-F, epicardial anchor device 300 allows for the epicardial anchor device to receive the tether T and to lock the tether (e.g. via pinning) at the desired tension without having to thread a free end of the tether T through the tether passageway 335. Rather, the epicardial anchor device 300 may be slid toward the tether T with the tether entering the epicardial anchor device 300 through the radial slot. It should also be understood that the steps shown in FIGS. 5C-E may be reversed to remove the tether T from the epicardial anchor device 300 via sliding. In other words, if the epicardial anchor device 300 is already pinned to tether T in the locked condition, the hub 350 may be rotated in the opposite direction relative to the base 340 and the pad assembly 320 to align the inner and outer radial slots 390a-b, and the epicardial anchor device 300 may be slid off the tether T without needing to pull the epicardial anchor device 300 along the length of the tether T. As used herein, the phrase unlocked condition generally refers to the condition in which the inner and outer radial slots 390a-b of the epicardial anchor device 300 are continuous, while the phrase locked condition generally refers to the condition in which the inner and outer radial slots of the epicardial anchor device are discontinuous. However, it should be understood that, when the epicardial anchor device 300 is engaged to the tether T in the locked condition, the epicardial anchor device is prevented from sliding away from the tether, both proximally and laterally relative to the tether. On the other hand, when the epicardial anchor device 300 is in the unlocked condition, the epicardial anchor device is capable of sliding away from the tether T, either proximally and/or laterally relative to the tether.

It should be understood that a delivery device identical to delivery device 248 may be used to manipulate the epicardial anchor device 300, for example including positioning the epicardial anchor device 300 and/or rotating the hub 350 to transition the epicardial anchor device 300 from the locked condition to the unlocked condition and vice versa. However, delivery device 248 may be modified in any suitable fashion to account for the radial slot in epicardial anchor device 300 that is not part of epicardial anchor device 200. In other embodiments, any suitable device may be used to manipulate the epicardial anchor device 300, including for example forceps (or any similar tool) to grasp the flat cutout sections of the base 340 (labelled as cutout section 266 in FIG. 3B for epicardial anchor device 200) and tweezers to rotate the hub 350 relative to the base 340. However, any system that is able base 340 relatively stationary while the hub 350 is rotated may be suitable. It should be understood that the hub 350 may include flat areas (as shown in FIGS. 5A and 5C-E, but not shown in FIGS. 5B and 5F) to assist with gripping the hub 350 in order to rotate the hub.

It should be understood that epicardial pad 300 may be simpler to both slide onto the tether as well as to slide off the tether compared to epicardial pad 200. Thus, epicardial pad 300 may be useful in an original implantation of a prosthetic heart valve PMV that includes a tether T, as well as for use in either (i) replacement or a previously implanted epicardial anchor device or (ii) re-tensioning a tether of a previously implanted prosthetic heart valve system without replacing the original epicardial anchor device. Because a free end of the tether T does not need to be threaded through a center of the epicardial anchor device 300, the time required to position the epicardial anchor device 300 against the epicardial surface with the tether received within the epicardial anchor device may be reduced due to the radial slot configuration. In addition to reducing the time required to position the epicardial anchor device 300 against the epicardial surface while the tether T is received within the epicardial anchor device, the radial slot may also reduce the complexity of the procedure. For example, after positioning of the heart valve, but before inserting the tether into the epicardial pad, the surgeon (or other surgical personnel) may need to manually hold the tether to ensure the prosthetic valve stays in place while the tether is coupled to the epicardial pad. By reducing the amount of time that the tether may need to be manually held prior to coupling to the epicardial pad, the chance of complications occurring from the manual holding of the tether may be reduced. The reduction in time and complexity may be beneficial to both the user and the patient, and further may help reduce the risk of the prosthetic heart valve PMV changing positions relative to the native valve annulus during the time of the procedure prior to the epicardial anchor device being locked to the tether at the desired tether tension.

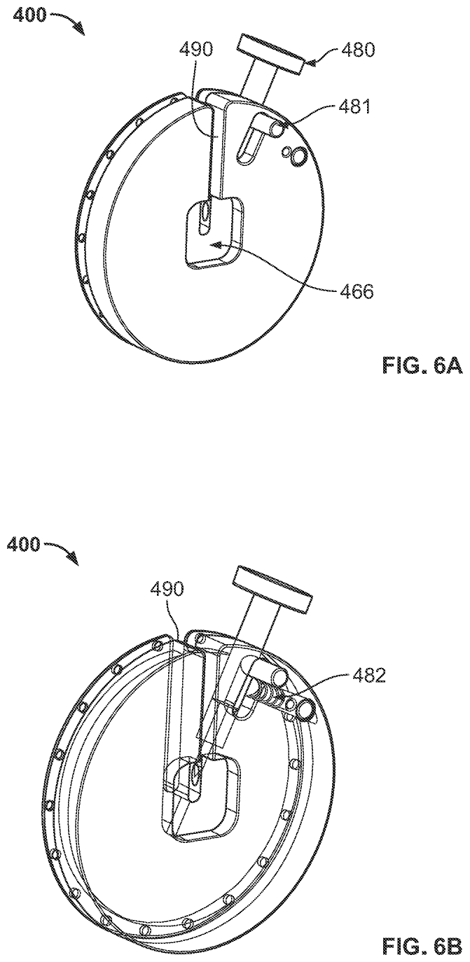

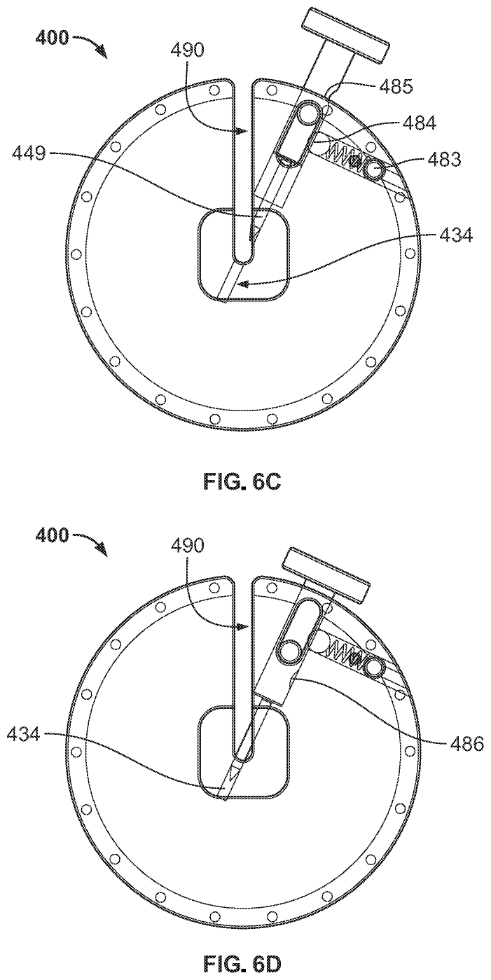

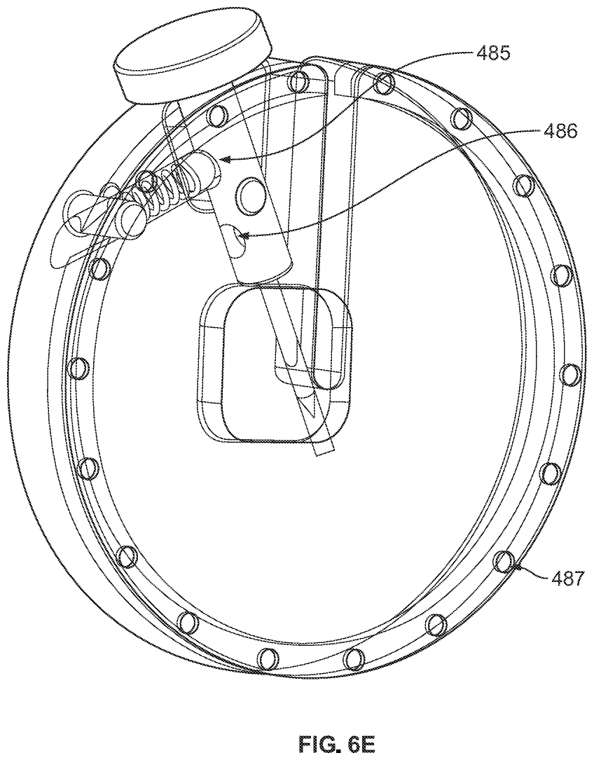

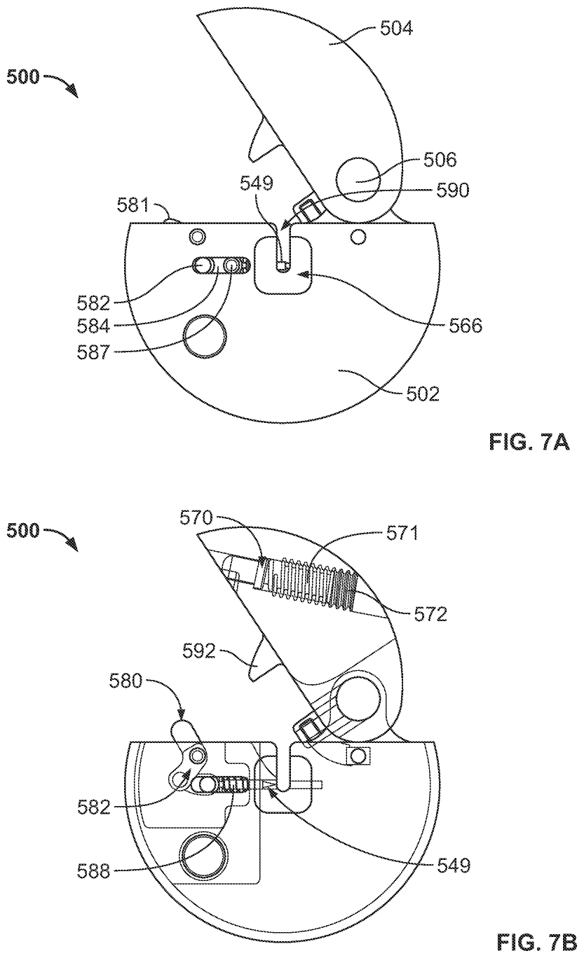

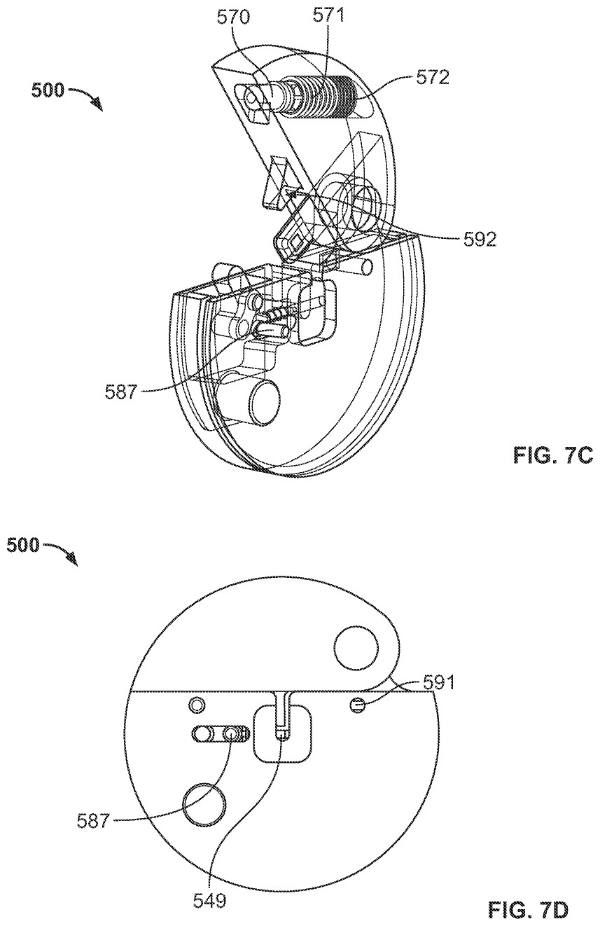

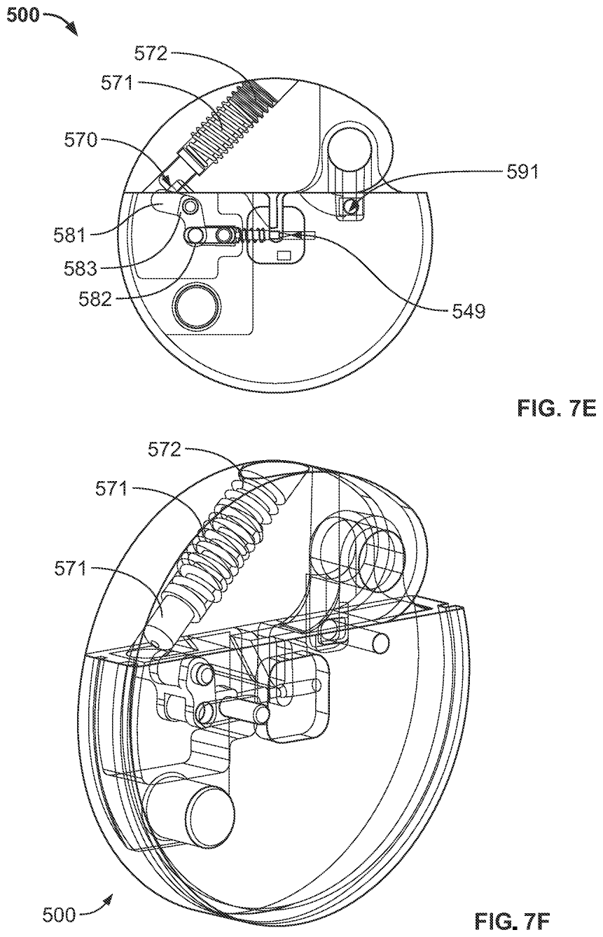

Although some embodiments of epicardial anchors with radial slots are described above, it should be understood that epicardial anchors may include radial slots with other designs than described above. These other embodiments, including those described below in connection with FIGS. 6A-7F, may provide overall similar benefits to the epicardial anchors described above. For example, the inclusion of a radial slot may decrease the amount of time required to lock the tether at the desired tension, and thus reduce the risk of procedural complications occurring prior to the tether being locked. And as with the embodiments described above, the embodiments described below may be suitable for an initial implantation, as well as a replacement of a previously implanted epicardial anchor.

FIGS. 6A-E illustrate various views of an epicardial anchor device 400 according to another aspect of the disclosure. Epicardial anchor device 400 may include a main body, which may be substantially circular, although it may take other shapes if desired. The main body of epicardial anchor device may be a single solid member, two opposing casing members that fit together with one another, or any other suitable construction. Epicardial anchor device 400 includes a radial slot 490 that has a first closed end positioned at or near the radial center of the epicardial anchor device, and that extends linearly to an outer perimeter of the housing, such that the radial slot 490 interrupts the otherwise continuous outer circumference of the epicardial anchor device 400. Unlike epicardial anchor device 300, the radial slot 490 of epicardial anchor device 400 may be formed of a single slot that has a fixed position relative to the housing of the epicardial anchor device 400.

Epicardial anchor device 400 may include a mechanism that allows for fixation or locking, for example by pinning, a tether that has been introduced via the radial slot 490 to a central portion of the epicardial anchor device 400. Epicardial anchor device 400 is illustrated in the unpinned condition in FIGS. 6A-C. Referring generally to FIGS. 6A-C, the locking pin assembly of epicardial anchor device 400 may be based on a pin-detent mechanism. For example, the locking pin 449 may include a leading piercing end and a trailing end that is fixed to a portion of pin knob 480. Pin knob 480 may include an enlarged trailing end, for example with a button shape, and a shaft extending from the enlarged trailing end. The shaft of the pin knob 480 may extend into a complementary channel within epicardial anchor device 400 so that the pin knob 480, and particularly the shaft thereof, is axially translatable to drive the locking pin 449 toward, or away from, the radial center of the epicardial pad 400 and/or the closed end of the radial slot 490. In the unpinned condition shown in FIGS. 6A-C, the pin knob 480 is at a maximum distance away from the radial center of the epicardial anchor device 400.

The shaft of the pin knob 480 may include a rod 481 extending outwardly therefrom, for example generally perpendicular to the longitudinal axis of the shaft of the pin knob 480. The rod 481 may be fixed to the shaft of the pin knob 480, and may extend through a generally oblong aperture in one housing face of the epicardial anchor device 400. In the unpinned condition shown in FIGS. 6A-C, the rod 481 is positioned at one end of the oblong aperture relatively far away from the radial center of the epicardial anchor device 400, and in the pinned condition shown in FIGS. 6D-E, the rod 481 is positioned at the opposite end of the oblong aperture relatively close to the radial center of the epicardial anchor device 400. The rod 481 may help define the limits to which the pin knob 480 may travel toward or away from the radial center of the epicardial anchor device 400, and may also provide a structure that tool may grip to push the pin knob 480 toward, or pull the pin knob 480 away from, the radial center of the epicardial anchor device 400. Such a tool may be used to do “fine tuning” of the tension on the tether in a second stage, after the user manually locks the epicardial anchor device 400 to the tether with pin knob 480 in a first stage. This is described in greater detail below.

The epicardial anchor device 400 may include a biasing member 482, such as a spring, having a first end in contact with a support 483. The support 483, which may be a rod or any suitable support structure, has a fixed position relative to the housing. A ball 484 may be positioned at a second end of the biasing member 482. The biasing member 482 may be positioned within a channel of the epicardial anchor device 400 that opens to the shaft of the pin knob 480, and the biasing member 482 may be in a compressed condition so that the biasing member 482 always exerts force to press the ball 484 toward the shaft of the pin knob 480. The shaft of the pin knob 480 may include a first detent 485 spaced away from a second detent 486. The two detents 485, 486 may both be positioned to face the ball 484, and may be have shapes sized to receive the ball 484 at least partially therein. The first detent 485 is positioned relatively far from the radial center of the epicardial anchor device 400 compared to the second detent 486. When the epicardial anchor device 400 is in the unpinned condition, shown in FIGS. 6A-C, the biasing member 482 presses the ball 484 into the second detent 486, tending to maintain the pin knob 480 in the unpinned condition. As shown in FIGS. 6D-E, when the epicardial anchor device 400 is in the pinned condition, the biasing member 482 presses the ball 484 into the first detent 485, tending to maintain the pin knob 480 in the pinned condition.

In use, the epicardial anchor device 400 may be initially in the unpinned condition. After positioning the prosthetic mitral valve PMV within the desired position in the heart, and with the tether T extending out of the patient's heart and being held by a surgeon or other personnel, the tether T may be slid to the center of the radial slot 490 laterally. When the tether T passes through the closed end of the radial slot 490 at or near the radial center of the epicardial anchor device 400, the epicardial anchor device 400 may be placed into contact with the outer surface of the heart. With at least some minimal tension applied to the tether T, which may be manually applied, the user may depress pin knob 480 to drive the leading, piercing end of the locking pin 449. As the leading, piercing end of the locking pin 449 is driven forward, it traverses the radial slot 490 near its closed end while passing through the tether T received therein. The locking pin 449 is driven until the pin knob 480 is advanced far enough that the ball 484 engages the first detent 485, and the leading, piercing end of the locking pin 449 is received within a locking pin channel 434 on the opposite side of the radial slot 490. The locking pin channel 434 may be sized and shaped to receive the leading, piercing end of the locking pin 449, as shown in FIG. 6D. The locking pin channel 434, and particularly the structure that defines the locking pin channel 434, may help to support and buttress the leading, piercing end of the locking pin 449 received therein. When in this pinned condition, the pin knob 480 is maintained in the desired axial position by the force of the biasing member 482 pressing the ball 484 into the first detent 485, with the locking pin channel 434 helping to support the locking pin 449. This may be referred to as a first phase pinning, since the main goal at this point at time may be to ensure quick connection of the epicardial anchor device 400 to the tether T, for example to minimize the amount of time the prosthetic mitral valve PMV is deployed within the native mitral valve annulus with the tether T being manually held by a surgeon or other operator. As noted elsewhere herein, the time during which tether T is manually held while the prosthetic mitral valve PMV is deployed may be a time in which complications have a relatively higher likelihood of occurring. Thus, the first phase of pinning the tether T to the epicardial anchor device 400 is preferably performed relatively quickly to ensure the prosthetic mitral valve PMV is secured after deployment, even if the tether T has not been fine-tuned to the precise desired tension.

After the tether T is pinned to the epicardial anchor device 400 in the first phase of pinning, the next phase of fine-tuning the tension of the tether T may begin. For example, the free end of the tether T may be passed into or through a tensioning tool and be pinned or otherwise fixed to the tensioning tool, with a distal end of the tensioning tool being received within receiver 466. The tensioning tool may also include a rotatable member with a curved channel which may receive rod 481 therein. The curvature of the channel may be generally similar to curved channel 260 shown in FIG. 3H. With (i) the distal end of the tensioning tool received within receiver 466; (ii) the rod 481 received within one end of the curved channel of the rotatable member of the tensioning tool; and (iii) the tether T pinned or otherwise fixed to the tensioning tool, the second phase fine-tuning of the tension of tether T may begin. That proves may include rotating the rotatable member of the tensioning tool to drive rod 481 away from the center of the epicardial anchor device 400, causing the piercing end of locking pin 449 to disengage the tether T. Because the tether T is now fixed to the tensioning tool, the prosthetic mitral valve PMV is not in danger of dislodging from the native mitral valve annulus. Further, the connection between the tensioning tool and the receiver 466 helps ensure the epicardial anchor device 400 stays in place and helps ensure that any forces applied to the epicardial anchor device 400 (e.g. the force applied to the rod 481) does not tend to move the main body of the epicardial anchor device 400 relative to the heart. With the rod 481 and locking pin 449 in the unlocked condition (e.g. as shown in FIG. 6C), the tensioning tool may be operated to tension the tether T to the desired force. Once the desired tension force is reached, the rotatable member of the tensioning tool may be rotated in the opposite direction as before, in order to drive the locking rod 481 (and thus locking pin 449) back into the locked position, piercing the tether T so that the tether T is locked at the desired tension. Then, the tensioning tool may be disengaged from the epicardial anchor device 400, and excess length of the tether T protruding beyond the epicardial anchor device 400 may be cut. It should be understood that the tensioning tool may have any suitable construction, including constructions described in connection with U.S. Patent Application Publication No. 2016/0367368, the disclosure of which is hereby incorporated by reference herein. And although the tensioning tool is described as having a rotatable mechanism with a curved channel to drive rod 481 between the locked (or pinned) and unlocked (or unpinned) conditions, other mechanisms may be similarly suitable.

Referring to FIG. 6E, epicardial anchor device 400 may include one or more through holes 487 extending through partially or fully through the housing of the epicardial anchor device 400. For example, in the illustrated embodiment, a plurality of through holes 487 are positioned at spaced distances around the circumference of the housing epicardial anchor device 400 near the outer perimeter thereof. These through holes 487 may serve to accept sutures or other fasteners therethrough. For example, as described above, it may be desirable for the epicardial anchor device 400, and particularly the face of the housing that will contact the heart, to have a fabric (e.g. velour) coupled thereto, for example to assist with quick sealing of the ventricular puncture which the tether T extends through, and for future tissue ingrowth to help to further fix the epicardial anchor device 400 to the heart. Such a fabric may be positioned on the desired face of the epicardial anchor device 400, and coupled to the epicardial anchor device 400 via sutures or other fasteners passing through the through holes 487.

FIGS. 7A-F illustrate various views of another embodiment of an epicardial anchor device 500. Referring to FIGS. 7A-B, epicardial anchor device 500 may include a lower housing 502 hingedly coupled to an upper housing 504, for example via a pin 506 or other hinge mechanism. Lower housing 502 and upper housing 504 may each form a portion of a circular shape, so that when epicardial anchor device 500 is in a closed condition, for example shown in FIG. 7D, the epicardial anchor device 500 is substantially circular. However, other shapes may be suitable and it should be understood that epicardial anchor device 500 need not be perfectly circular when in the closed condition. Each of the lower housing 502 and upper housing 504 may include a contoured or partially circular portion, and a substantially straight portions, with the substantially straight portions confronting each other when the epicardial anchor device 500 is in the closed condition. The lower housing 502 may include a radial slot 590 extending from near the radial center of the epicardial anchor device 500 to the straight edge portion of the lower housing 502, so that when the epicardial anchor device 500 is in the open condition, a tether T may be slid laterally along the radial slot 590 until the tether T is positioned at or near the radial center of the epicardial anchor device 500 (and/or adjacent the closed end of the radial slot 590).

Referring to FIGS. 7B-C and 7E-F, the upper housing 504 may include a channel that receives a pressure pin 570. As best shown in FIG. 7B, the channel may have a shoulder and the pressure pin 570 may have a head that contacts the shoulder but cannot pass through the shoulder. The narrower end of pressure pin 570 pass through the channel so that it may contact actuator 580 when the epicardial anchor device 500 is in the closed condition. The channel that receives the pressure pin 570 may be threaded along some length thereof, and a set screw 572 may be received in the channel and engage the threads. A biasing member, such as a spring 571 may be positioned between the set screw 572 and the pressure in 570, with one end of the spring 571 pressing against the head of the pressure pin 570. With this configuration, the set screw 572 may be rotated to translate the set screw 572 toward or away from the pressure pin 570 to compress or relax the spring 571, respectively, to increase or decrease the amount of force applied to the pressure pin 570. As is described in greater detail below, this configuration helps to allow the epicardial anchor device 500 to be temporarily switched to the unpinned condition, even when the epicardial anchor device 500 is in the closed condition.

The pinning mechanism of epicardial anchor device 500 may include an actuator 580, which in the illustrated embodiment may be a bellcrank actuator. In the illustrated embodiment, actuator 580 may be generally “V”-shaped, although other shapes may be suitable. For example, actuator 580 may have a top portion 581 that may protrude beyond and/or extend above the straight edge of lower housing 502, and an opposite bottom portion 582 that extends into lower housing 502. A central portion 583 of actuator 580 may be hingedly coupled to lower housing 502, for example via a pin extending through an aperture of the central portion 583. With this configuration, the actuator 580 is rotatable about the central portion 583. A locking pin 549 may be coupled to a pin driver 584. In some embodiments, the pin driver 584 may be positioned in contact with the bottom portion 582 of the actuator 580, so that as the bottom portion 582 of the actuator 580 rotates, it pushes the pin driver 584 to advance the locking pin 549. In other embodiments, the pin driver 584 may be coupled to the bottom portion 582 of the pin driver 584. For example, the bottom portion 582 of the actuator 580 may include one or more apertures, and the pin driver 584 may include a pin or rod received within the one or more apertures. However, it should be understood that other types of coupling mechanisms may be used if the bottom portion 582 of the actuator 580 is coupled to the pin driver 584 to assist in driving the pin driver 584 upon rotation of the actuator 580.

Referring to FIG. 7B, a biasing member, such as a spring 588, may bias the pin driver 584 in a direction away from the slot 590. In the particular illustrated example, spring 588 is a coil spring that receives a portion of the locking pin 549 therethrough, with one end of the spring 588 in contact with pin driver 584, and the opposite end of the spring 588 in contact with a surface of lower housing 502 in which the pinning mechanism is received. The pin driver 584 may include a rod 587 extending therefrom, the rod 587 passing through a slot within the lower housing 502 so that the rod 587 may be manipulated from outside the epicardial anchor device.

In the unpinned condition of epicardial anchor device 500, for example as shown in FIG. 7B, the spring 588 biases the pin driver 584 so that the piercing end of locking pin 549 is positioned on one side of slot 590 so that the locking pin 549 does not traverse the slot 590. In order to transition the epicardial anchor device 500 from the unpinned condition to the pinned condition, the upper housing 504 may be closed by rotating it about pin 506. As the upper housing 504 closes, the leading end of pressure pin 570 contacts the top portion 581 of actuator 580, forcing the actuator 580 to rotate about the central portion 583 of the actuator 580. As the actuator 580 rotates, the bottom portion 582 of the actuator 580 drives the pin driver 584 linearly, compressing spring 588 as the piercing end of the locking pin 549 passes through the slot 590. The pinned condition is shown in FIGS. 7D-F. In the pinned condition, the leading end of locking pin 549 is received within a pin channel on the opposite side of the slot 590 from the rest of the pinning mechanism, the pin channel helping to stabilize the leading end of the locking pin 549. The epicardial anchor device 500 may remain in the closed condition via any suitable mechanism, preferably one that is reversible. For example, as shown in FIG. 7E, the upper housing 504 may include a segment with a detent 591, which may be a member having some amount of compressibility or flexibility. As the upper housing 504 rotates towards the closed condition, the detent 591 may ride along a complementary channel in the lower housing 502, until the detent 591 reaches an opening at the end of the complementary channel. Upon reaching that opening, the detent 591 may decompress or flex so that it “snaps” into place through the opening, effectively locking the epicardial anchor device 500 in the closed condition. If it becomes desirable to transition the epicardial anchor device 500 back to the open condition, a user may depress the detent 591 while rotating the upper housing 504 away from the lower housing 502.

While the epicardial anchor device 500 is locked within the closed condition, spring 571 and set screw 572 provide a force on pressure pin 570 to maintain the actuator 580 in the actuated condition, despite the opposite force provided by compressed spring 588. In other words, the force of spring 571 is greater than the force of spring 588 to keep the locking pin 549 in the pinned condition when the epicardial anchor device 500 is in the closed condition.

As best illustrated in FIGS. 7A-C, the upper housing 504 may include a protrusion 592 extending downward from the straight edge thereof so that, as the upper housing 504 closes on the lower housing 502, the protrusion 592 extends into the slot 590, as best illustrated in FIG. 7D. The lower housing 502 may include a ramped surface that leads into slot 590, with the protrusion 592 and ramped surface having generally complementary shapes. With this configuration, while the tether is positioned within slot 590 and the epicardial anchor device 500 is transitioned from the open condition to the closed condition, the protrusion 592 rides along the corresponding ramped surface, forcing the tether T to be pushed toward the closed end of the slot 590 in the path of the locking pin 549 as the locking pin 549 transitions to the pinned condition.

Epicardial anchor device 500 may be used in a generally similar method as described above in connection with epicardial anchor device 400. In use, the epicardial anchor device 500 may be initially in the open and unpinned condition. After positioning the prosthetic mitral valve PMV within the desired position in the heart, and with the tether T extending out of the patient's heart and being held by a surgeon or other personnel, the tether T may be slid toward the center of the radial slot 590 laterally. When the tether T passes through the closed end of the radial slot 590 at or near the radial center of the epicardial anchor device 500, the epicardial anchor device 500 may be manually closed by rotating the upper housing 504 toward the lower housing 502 until the detent 591 clicks into place. It should be understood that, even if the tether T is not directly in closed end of slot 590 during closing of the epicardial anchor device 500, the protrusion 592 will help push or otherwise guide the tether T toward the closed end of the radial slot 590. As this closing occurs, the pressure pin 570 actuates the actuator 580, driving in the locking pin 549 through the tether T to pin the tether T to the epicardial anchor device 500. Preferably, this simultaneous closing and pinning is performed with at least a minimal amount of tension manually applied to the tether T. As described above, the detent 591 maintains the epicardial anchor device 500 in the closed condition, while the force of the spring 571 ensures that the epicardial anchor device 500 stays in the pinned condition in the absence of additional applied forces. This initial closing and pinning may be referred to as a first phase, since the main goal at this point at time may be to ensure quick connection of the epicardial anchor device 500 to the tether T, for example to minimize the amount of time the prosthetic mitral valve PMV is deployed within the native mitral valve annulus with the tether T being manually held by a surgeon or other operator. As noted elsewhere herein, the first phase of pinning the tether T to the epicardial anchor device 500 is preferably performed relatively quickly to ensure the prosthetic mitral valve PMV is secured after deployment, even if the tether T has not been fine-tuned to the precise desired tension.

After the tether T is pinned to the epicardial anchor device 500 in the first phase of pinning, a second phase of fine-tuning of the tension T may be performed in much the same manner as described above in connection with epicardial anchor device 400. For example, the free end of the tether T may be passed into and temporarily fixed to a tether tensioning tool that has a distal end positioned within receiver 566 and an actuator (e.g. a rotatable actuator with a curve channel) that receives rod 587 therein. The actuator of the tether tensioning tool may be actuated (e.g. rotated) to drive the rod 587 away from the tether T, resulting in the spring 588 relaxing and the actuator 580 rotating to force pressure pin 570 upward while compressing spring 571. Importantly, the set screw 572 should be positioned so that spring 571 produces enough force to keep actuator 580 actuated when additional forces are not applied, but not so much force as to restrict the rod 587 from being able to be transitioned to temporarily unpin the tether T during this second stage of fine-tuning. With the epicardial anchor device 500 still closed, but in the unpinned condition, the tension of the tether T may be fine-tuned to the desired tension. When at the desired tension, the actuator of the tensioning tool may again be actuated (e.g. rotated in the opposite direction) to allow the spring 571 to force pressure pin 570 into the actuator 580 to again drive the locking pin 549 through the tether T, locking the tether T at the fine-tuned tension. The tensioning tool may then be removed and the procedure completed.

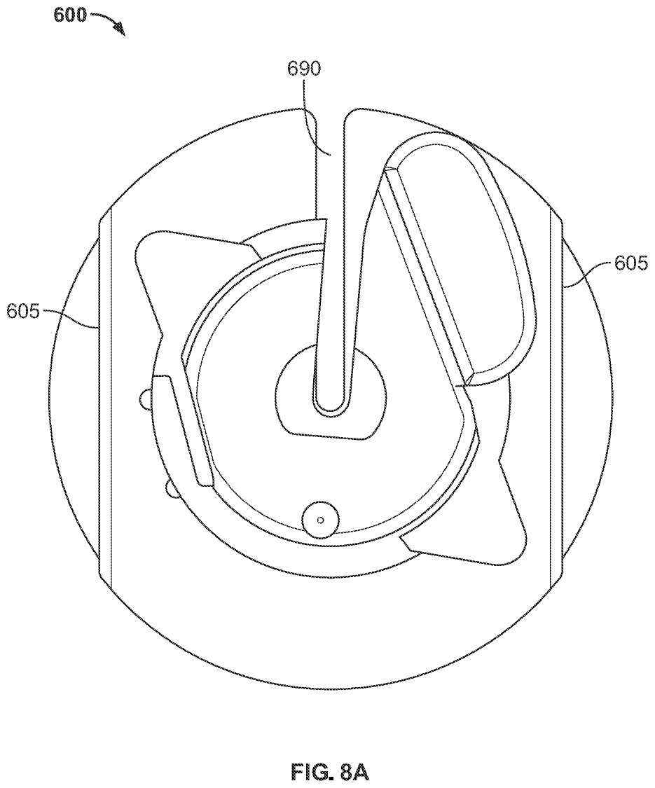

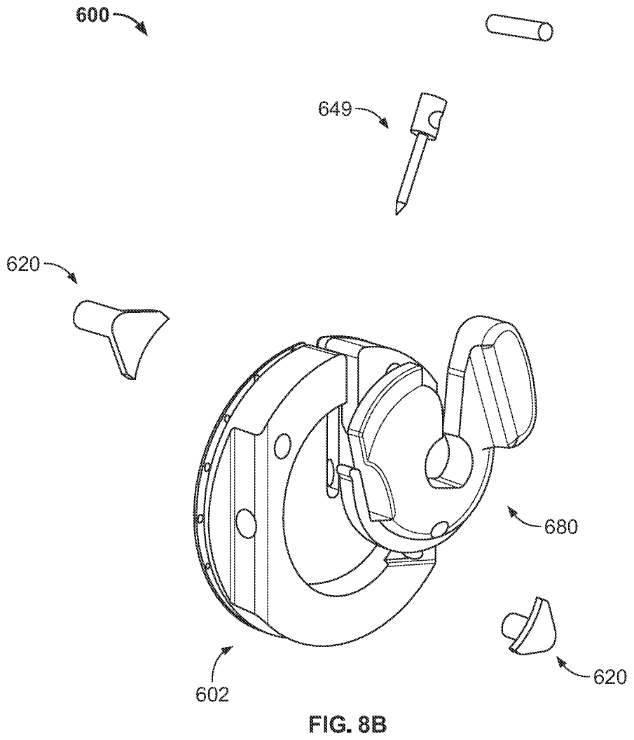

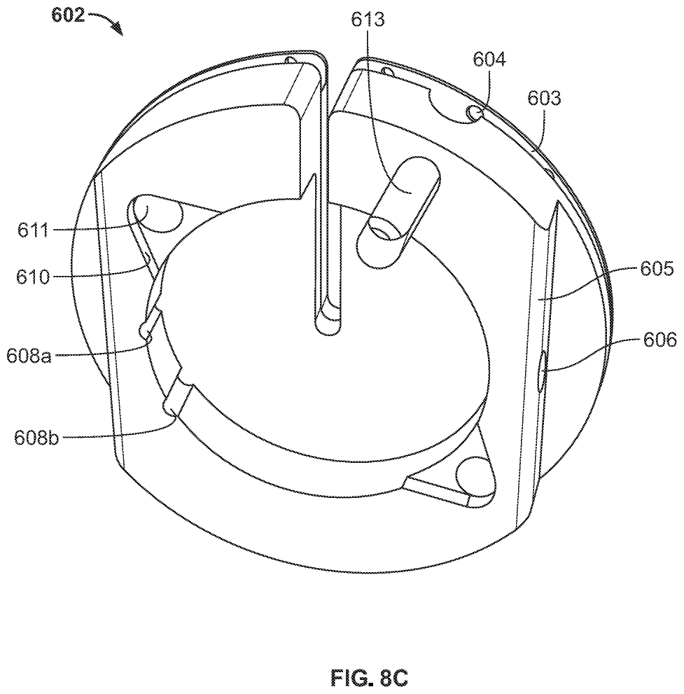

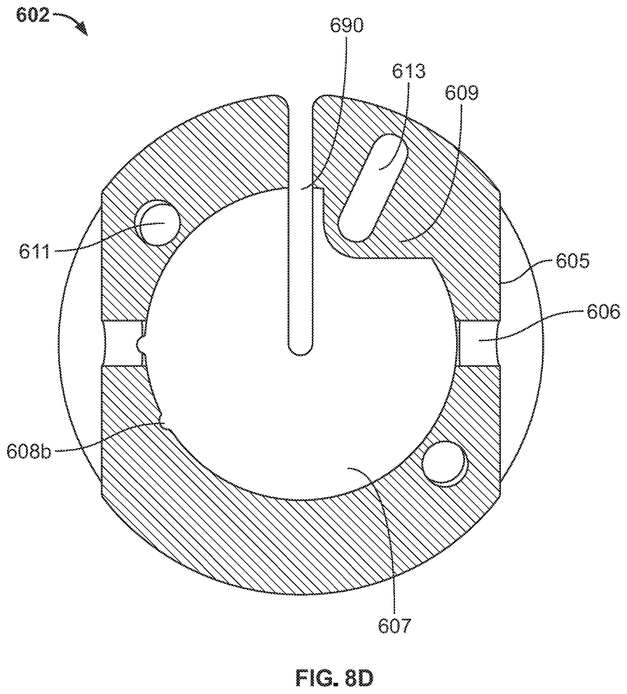

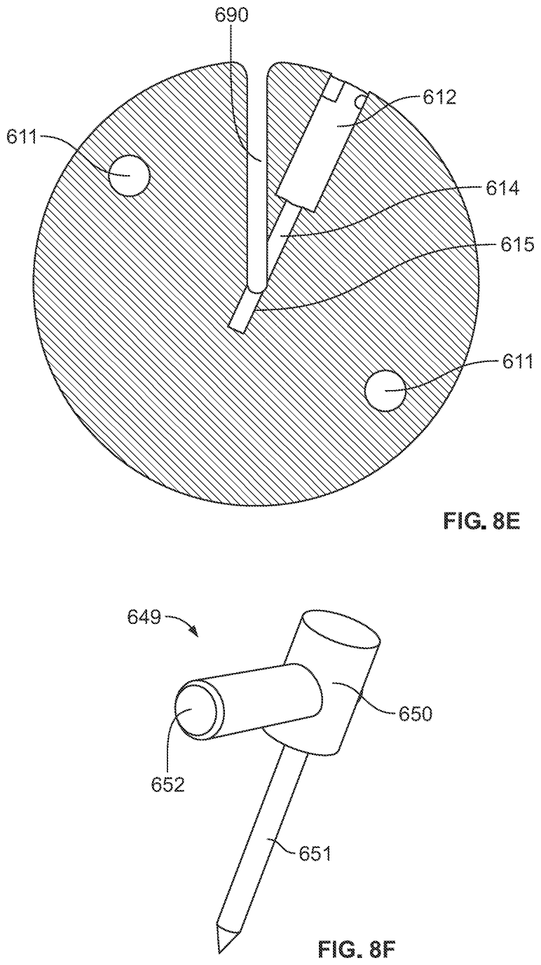

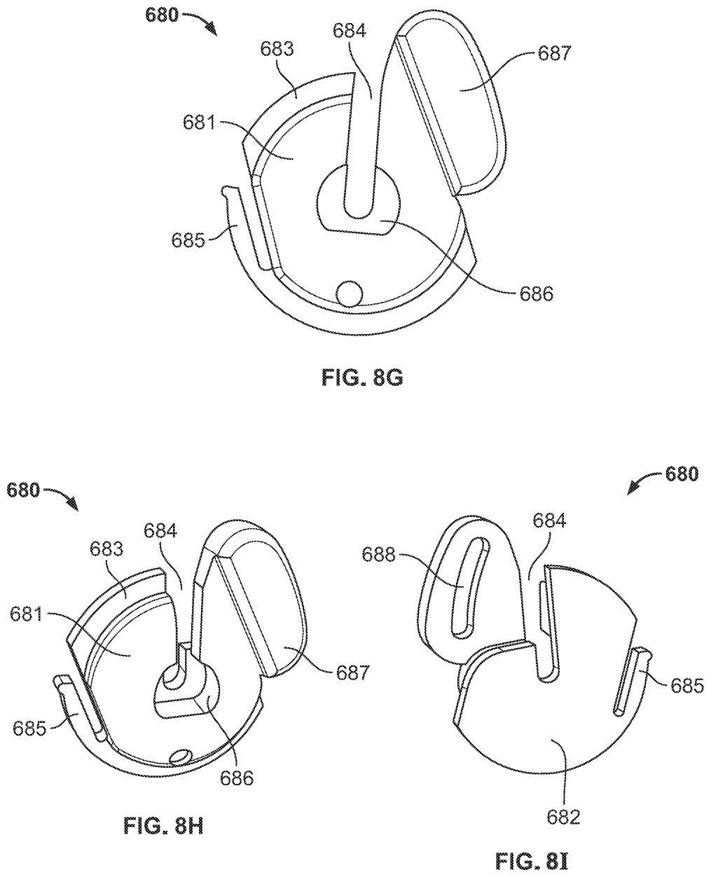

FIG. 8A is a front view of an epicardial anchor device 600 according to another embodiment of the disclosure. Generally, as best shown in the exploded view of FIG. 8B, epicardial anchor device 600 may include a base 602, an actuator (which may also be referred to as a hub) 680, one or more actuator retainers 620, and a locking pin 649.

The base 602 is illustrated in perspective and cross-sectional views in FIGS. 8C and 8D, respectively. The base 602 may have a generally round or circular rear section 603, which may include one or more apertures 604. Apertures 604 may be used to fix another member, such as a synthetic fabric, to the rear section 603. The rear section 603 is intended to directly contact the heart tissue, and the synthetic fabric may assist with sealing the transapical puncture, including via clotting and tissue ingrowth into the fabric.

Base 602 has a front section that may include two opposing flats 605. Each flat 605 may include an aperture defining a channel 606 extending radially inwardly. These flats 605 and channels 606 may be substantially similar to those of base member 240, and may be configured for receiving complementary gripping features of a tether tensioning tool, including tools similar or identical to those described in U.S. Patent Application Publication No. 2016/0367368, the disclosure of which is hereby incorporated by reference herein. For example, the tether tensioning tool may be similar to delivery device 248 shown and described in connection with FIG. 4.

Referring to FIGS. 8C-D, the front portion of base 602 may define a generally cylindrical recess 607. The wall defining recess may be interrupted by two (or more) cutouts or detents 608a, 608b as best shown in FIG. 8C. Cutouts or detents 608a, 608b may have generally similar functionality as cutouts or detents 243 of base 240, and the function of detents 608a, 608b is described in greater detail below. The wall defining cylindrical recess 607 may also be interrupted by radial slot 690. The wall defining cylindrical recess 607 may also include a protrusion 609 that extends radially inwardly toward the center of the cylindrical recess 607.

Referring to FIG. 8C, the base 602 may include one or more retainer recesses 610 positioned at or adjacent the cylindrical recess 607. In the illustrated embodiment, base 602 includes two generally triangular shaped retainer recesses 610 positioned at diametrically opposed locations of the cylindrical recess 607. However, it should be understood that more or fewer retainer recesses 610 may be provided, at similar or different locations than shown, and the retainer recesses 610 may have other shapes. As is explained in greater detail below, retainer recesses 610 may be shaped and sized to receive actuator retainers 620 therein. Apertures 611 may extend through the front of rear of base 602 in the retainer recesses 610, and may be configured to receive portions of the actuator retainers 620 therein to help secure the actuator retainers 620 to the base 602.

Referring to FIGS. 8D-8E, additional structures of base 602 for receiving the locking pin 649 (or components thereof) are shown. As shown in FIG. 8E, a locking pin channel may be formed in base 602, the locking pin channel including a larger diameter portion 612 that opens to the exterior of the base 602, and a smaller diameter portion that traverses the radial slot 690. The smaller diameter portion of the channel may include a proximal portion 614 that is opens to the larger diameter portion 612, and a distal portion 615 positioned on the other side of the radial slot 690, the distal portion terminating at a closed end. Briefly referring to FIG. 8F, the locking pin 649 may include an enlarged head portion 650 sized and shaped to be relatively tightly received within the larger diameter portion 612, and a smaller shaft portion 651, which may include the leading piercing end, that is sized and shaped to be relatively tightly received within the smaller diameter portions 614, 615. It should be understood that although the term “relatively tightly” is used, the locking pin 649 should be capable of translation within the locking pin channel.

Referring to FIGS. 8C-D, the front face of base 602 may include an oblong slot 613 that leads to the larger diameter portion 612 of the locking pin channel. This oblong slot 613 is sized to receive rod 652 of locking pin 649 therethrough. Referring briefly to FIG. 8F, rod 652 may be a generally cylindrical member coupled to and extending from the enlarged head 650 of the locking pin 649, and the rod 652 may extend in a direction substantially orthogonal or perpendicular to the long axis of the locking pin head 650 and shaft 651. Referring back to FIGS. 8C-D, the oblong slot 613 may be closed at each terminal end and may be generally positioned in or near protrusion 609. The oblong slot 613 primarily functions to allow a user to manipulate rod 652 during the second stage of fine tuning of the tension of tether T, but may also assist in guiding the translation of the locking pin 649 and limiting the extent to which the locking pin 649 is capable of translating within the locking pin channel.

FIGS. 8G-I illustrate different views of actuator 680. Generally, actuator 680 may include a front face 681 and a rear face 682, the rear face configured to be received within cylindrical recess 607 and confront base 602, with the front face 681 being accessible in the assembled condition of the epicardial anchor device 600. The front face 681 may protrude a distance from the rear face 682 so as to define a reduced thickness rim 683, the rim 683 generally following along the arc of a circle, although not all 360 degrees of a circle. When the actuator 680 is received within the base 602, and the actuator retainers 620 are received within retainer recesses 610, the actuator retainers 620 overlie portions of the rim 683 to help retain the actuator 680 connected to the base 602, while still allowing for an amount of rotation of the actuator 680 relative to the base. This relative positioning is best shown in FIG. 8A. However, it should be understood that other mechanisms may be used to rotatably retain the actuator 680 within (or coupled to) base 602.

Referring again to FIGS. 8G-I, actuator 680 may include a radial slot 684 extending from a radial center of the actuator to and through a portion of rim 683. When the actuator 680 is received within the base 602 in an unpinned or unlocked condition, in the position illustrated in FIG. 8A, the radial slot 684 of the actuator 680 aligns with the radial slot 690 of the base 602, so that a tether T may be laterally slid along both radial slots simultaneously until the tether T is positioned at a radial center of the epicardial anchor device 600. A portion of the rim 683 may include an arm 685 capable of flexing, for example by virtue of a slot created between the arm 685 and the rest of the structure forming the front face 681 and rear face 682 of actuator 680. Arm 685 may include a tip with a protrusion that is sized and positioned to be received within detent 608a or detent 608b depending on the rotational position of the actuator 680 relative to the base 602. For example, in the unpinned or unlocked condition shown in FIG. 8A, the tip of arm 685 may be received within detent 608a. However, following counterclockwise rotation of the actuator 680 relative to the base 602, the tip of arm 685 may exit detent 608a, assisted by the flexible nature of the arm 685, until the tip of the arm 685 reaches detent 608b and “snaps” into that detent. Preferably, arm 865 has enough spring force to help maintain actuator 680 in a current rotational position relative to the base 602 when the tip of the arm 685 is received within either detent 608a or 608b, but not so much force as to make it difficult to intentionally rotate actuator 680. In other words, the configuration of arm 685 and detents 608a, 608b help avoid unintentional rotation of actuator 680, without hindering intentional rotation of actuator 680, while also providing feedback to the user as the actuator 680 rotates between the two discrete locked and unlocked conditions.

Referring now to FIGS. 8G-H, the front face 681 of actuator 680 may include a non-circular recess 686 at a radial center thereof, the recess 686 extending toward, but not through, the rear face 682. This recess 686 may be generally similar to the recess shown in FIG. 3G for hub 250. In other words, the recess 686 may be shaped and positioned to receive the leading end portion of a tether tensioning tool, such as that shown in FIG. 4, such that rotation of the leading end portion of the tether tensioning tool forces corresponding rotation of the actuator 680 when the tether tensioning tool is engaged to the actuator 680. It should also be understood that, when the tether tensioning tool is engaged with the recess 686 of actuator 680, the tether T may extend through a shaft of the tether tensioning tool and be pinned to a member thereof, for example a member including a force gauge, so that the tether may be manipulated to a particular desired tension while pinned to the tether tensioning tool.

Referring again to FIGS. 8G-I, the actuator 680 may include an extension 687 extending therefrom, for example generally diametrically opposed the arm 685. The rear face of the extension 687, as best shown in FIG. 8I, may include a channel 688 therein, which may be a generally curved channel. Preferably, the curvature of the channel 688 is generally similar to that of 260, at least in the sense that the channel is not a constant arc of a circle, but rather has a curvature that allows for cam action of the channel 688 as the actuator 680 is rotated. In the assembled condition of the epicardial anchor device 600, the rod 652 of locking pin 649 is received within channel 688, for example at or near the top end of the channel in the view of FIG. 8I. In the view of FIG. 8I, the top end of the channel 688 is positioned a greater distance from a radial center of the actuator 680 than is the opposite bottom end of the channel With this configuration, a user may manually grip extension 687 and rotate the actuator 680 counterclockwise relative to the base 602 (in reference to the view of FIG. 8A) to cause the rod 652 to slide along the channel 688. As the rod 652 traverses the channel 688, the rotational movement of the actuator is translated to axial movement of the rod 652, causing the locking pin 649 to drive toward ad through radial slot 690 until the leading piercing end of the locking pin 649 is located in the distal portion 615 of the locking pin channel. It should be understood that, when the epicardial anchor device 600 is in the locked or pinned condition, although the closed ends of radial slots 684 and 690 may align with each other, the remaining portions of the radial slots 684 and 690 may not align with each other. Thus, in the unlocked condition, the two radial slots 684, 690 may provide for a continuous pathway from the radial center to and through the outer circumference of the epicardial anchor device 600. However, in the locked or pinned condition, the radial slots 684 and 690 may be offset so there is no continuous pathway from the radial center to and through the outer circumference of the epicardial anchor device 600.

Epicardial anchor device 600 may be used in a generally similar method as described above in connection with epicardial anchor devices 400, 500. In use, the epicardial anchor device 600 may be initially in the open and unpinned condition, as illustrated in FIG. 8A. After positioning the prosthetic mitral valve PMV within the desired position in the heart, and with the tether T extending out of the patient's heart and being held by a surgeon or other personnel, the tether T may be slid toward the center of the radial slots 684 and 690 laterally. When the tether T passes through the closed ends of the radial slots 684 and 690 at or near the radial center of the epicardial anchor device 600, the epicardial anchor device 600 may be manually transitioned to the locked condition by manually rotating the extension counterclockwise until the protrusion on arm 685 is received within detent 608b. As described above, as this counterclockwise rotation of actuator 680 occurs, the movement of the channel 688 drives rod 652, and thus locking pin 649, along the locking pin channel and through the tether T to pin the tether T to the epicardial anchor device 600. Preferably, this first stage of pinning is performed with at least a minimal amount of tension manually applied to the tether T. As described above, the interaction between the protrusion of arm 685 and detent 608b maintains the epicardial anchor device 600 in the closed condition. This initial pinning may be referred to as a first phase, since the main goal at this point at time may be to ensure quick connection of the epicardial anchor device 600 to the tether T, for example to minimize the amount of time the prosthetic mitral valve PMV is deployed within the native mitral valve annulus with the tether T being manually held by a surgeon or other operator. As noted elsewhere herein, the first phase of pinning the tether T to the epicardial anchor device 600 is preferably performed relatively quickly to ensure the prosthetic mitral valve PMV is secured after deployment, even if the tether T has not been fine-tuned to the precise desired tension.

After the tether T is pinned to the epicardial anchor device 600 in the first phase of pinning, a second phase of fine-tuning of the tension T may be performed. For example, the free end of the tether T may be passed into and temporarily fixed to tether a tensioning tool, such as that shown in FIG. 4, with arms 268 engaging channels 606, and the distal tip of the tensioning tool received within recess 686. The distal tip of the tether tensioning tool may be rotated clockwise to drive the locking pin 649 away from the tether T, transitioning the epicardial anchor device back to the unlocked condition While the epicardial anchor device 600 is temporarily in the unpinned condition, the tension of the tether T may be fine-tuned to the desired tension using the tether tensioning tool. Then, when the tether T is at the desired tension, the distal tip of the tether tensioning tool may be rotated counterclockwise, to transition the epicardial anchor device 600 back to the locked or pinned condition, such that the locking pin 649 drives through the tether T to lock the tether T at the desired tension. The tensioning tool may then be removed and the procedure completed.

According to a first aspect of the disclosure, an epicardial anchor device comprises:

a base defining an outer radial slot; and

a hub defining an inner radial slot, the hub received at least partially within the base, the hub being rotatable about a center longitudinal axis relative to the base, the hub defining, at least in part, a tether passageway for receiving therethrough a tether coupled to a prosthetic heart valve, the tether passageway being continuous with the inner radial slot,

wherein the epicardial anchor device has an unlocked configuration in which the inner radial slot is discontinuous with the outer radial slot, and a locked configuration in which the inner radial slot is continuous with the outer radial slot, the epicardial anchor device being transitionable from the locked configuration to the unlocked configuration via rotation of the hub about the center longitudinal axis relative to the base in a first rotational direction, and transitionable from the unlocked configuration to the unlocked configuration via rotation of the hub about the center longitudinal axis relative to the base in a second rotational direction opposite the first rotational direction; and/or

the outer radial slot extends through an outer circumferential rim of the epicardial anchor device so that the outer circumferential rim is interrupted by the outer radial slot; and/or

a locking pin assembly disposed between the hub and the base; and/or

a bottom surface of the hub includes a curved channel therein, the bottom surface of the hub confronting a top surface of the base; and/or

a portion of the locking pin assembly is received within the curved channel; and/or

a piercing portion of the locking pin assembly is configured to drive through the tether passageway upon transitioning the epicardial anchor device from the unlocked configuration to the locked configuration; and/or

a pad assembly coupled to a bottom surface of the base; and/or

the pad assembly is formed of a fabric material, the pad assembly configured to contact a heart of the patient; and/or

the outer radial slot extends radially through the pad assembly; and/or

the epicardial anchor device may be part of a prosthetic heart valve system including the prosthetic heart valve and the tether, the tether having a first end coupled to the prosthetic heart valve and a second free end.

According to another aspect of the disclosure, a method of implanting a prosthetic heart valve system comprises:

implanting a prosthetic heart valve into a native heart valve annulus of a patient so that a tether has a first end fixedly coupled to the prosthetic heart valve and a second free end, an intermediate portion of the tether extending through a wall of a heart of the patient while the prosthetic heart valve is in the native heart valve annulus;

sliding an epicardial anchor device over the intermediate portion of the tether so that the intermediate portion of the tether passes through an outer radial slot in a base of the epicardial anchor device and through an inner radial slot in a hub of the epicardial anchor device, until the intermediate portion of the tether is received within a central tether passageway of the epicardial anchor device, the inner radial slot being continuous with the outer radial slot during the sliding;

positioning the epicardial anchor device in contact with the wall of the heart;

tensioning the tether to a desired tension level; and

while the tether is tensioned to the desired tension level, rotating the hub about a center longitudinal axis relative to the base so that the inner radial slot is discontinuous with the outer radial slot; and/or

rotating the hub about the center longitudinal axis relative to the base transitions the epicardial anchor device from an unlocked configuration to a locked configuration; and/or

transitioning the epicardial anchor device from the unlocked configuration to the locked configuration causes the tether to be maintained at the desired tension level; and/or

transitioning the epicardial anchor device from the unlocked configuration to the locked configuration causes a piercing portion of a locking pin assembly within the epicardial anchor device to drive through the central tether passageway and the intermediate portion of the tether.

According to another aspect of the disclosure, a method of adjusting a prosthetic heart valve system comprises:

accessing an epicardial anchor device after a first prosthetic heart valve implantation procedure has been performed, the first prosthetic heart valve implantation procedure having included implanting the prosthetic heart valve into a native valve annulus of a patient and coupling the epicardial anchor device to the prosthetic heart valve via a tether fixed to the prosthetic heart valve so that the epicardial anchor device is positioned on an epicardial surface of the heart;

after accessing the epicardial anchor device, gripping the tether;

while the tether is gripped, transitioning the epicardial anchor device from a locked condition to an unlocked condition by rotating a hub of the epicardial anchor device about a center longitudinal axis relative to a base of the epicardial anchor device so that an inner radial slot of the hub is continuous with an outer radial slot of the base; and

after transitioning the epicardial anchor device to the unlocked condition, sliding the epicardial anchor device away from the tether so that the tether passes from a central tether passageway of the hub through the inner and outer radial slots until the epicardial anchor device no longer receives the tether; and/or

the first prosthetic heart valve implantation procedure had included fixing the epicardial anchor device to the prosthetic heart valve via the tether while the tether is tensioned to a first amount of tension; and

accessing the epicardial anchor device is performed after the first amount of tension decreases over time to a second amount of tension smaller than the first amount of tension; and/or

the epicardial anchor device is a first epicardial anchor device; and

after sliding the first epicardial anchor device away from the tether so that the first epicardial anchor device no longer receives the tether, coupling a second epicardial anchor device to the tether, the second epicardial anchor device being different than the first epicardial anchor device; and/or

the first epicardial anchor device has a first surface area of contact with the epicardial surface of the heart, and the second epicardial anchor device has a second surface area of contact with the epicardial surface of the heart greater than the first surface area of contact; and/or

prior to sliding the first epicardial anchor device away from the tether, the epicardial surface of the heart includes a dimple at a location of the first epicardial anchor device, and the second surface area of contact is larger than a surface area of the dimple; and/or

tension is maintained on the tether between (i) transitioning the first epicardial anchor device to the unlocked condition and (ii) coupling the second epicardial anchor device to the tether.

According to a further aspect of the disclosure, an epicardial anchor device comprises:

a base defining a radial slot; and

a hub defining a radial slot, the hub received at least partially within the base, the hub being rotatable about a center longitudinal axis of the hub relative to the base, the radial slot of the hub defining, at least in part, a tether passageway for receiving therethrough a tether coupled to a prosthetic heart valve,

wherein the epicardial anchor device has an unlocked configuration in which the radial slot of the base aligns with the radial slot of the hub so that the tether may be slid laterally through the radial slot of the base and the radial slot of the hub to position the tether at a radial center of the hub, and a locked configuration in which the radial slot of the base is out of alignment with the radial slot of the hub so that the tether cannot be slid laterally away from the radial center of the hub, the epicardial anchor device being transitionable from the locked configuration to the unlocked configuration via rotation of the hub about the center longitudinal axis of the hub relative to the base in a first rotational direction, and transitionable from the unlocked configuration to the locked configuration via rotation of the hub about the center longitudinal axis of the hub relative to the base in a second rotational direction opposite the first rotational direction; and/or

a locking pin assembly disposed within the epicardial anchor device; and/or

a bottom surface of the hub includes a curved channel therein, the bottom surface of the hub confronting a top surface of the base; and/or

a rod of the locking pin assembly is received within the curved channel; and/or

a piercing portion of the locking pin assembly is configured to drive through the tether passageway upon transitioning the epicardial anchor device from the unlocked configuration to the locked configuration; and/or

a pad assembly coupled to a bottom surface of the base; and/or

the pad assembly is formed of a fabric material, the pad assembly configured to contact a heart of the patient; and/or

the radial slot of the base is an outer radial slot, and the radial slot of the hub is an inner radial slot; and/or

in the unlocked configuration of the epicardial anchor device, the inner radial slot is continuous with the outer radial slot; and/or

in the locked configuration of the epicardial anchor device, the inner radial slot is discontinuous with the outer radial slot; and/or

the outer radial slot extends through an outer circumferential rim of the epicardial anchor device so that the outer circumferential rim is interrupted by the outer radial slot; and/or

a locking pin assembly, the locking pin assembly including a locking pin disposed within a locking pin channel within the base, and a rod extending transversely from the locking pin, the rod protruding through the base; and/or

the hub includes an extension, a bottom surface of the extension defining a curved channel therein, the bottom surface of the extension confronting a top surface of the base; and/or

the rod of the locking pin assembly is received within the curved channel of the extension; and/or

the epicardial anchor device is part of a prosthetic heart valve system that includes the prosthetic heart valve and the tether, the tether having a first end coupled to the prosthetic heart valve and a second free end.

According to yet a further aspect of the disclosure, a method of implanting a prosthetic heart valve system comprises:

implanting a prosthetic heart valve into a native heart valve annulus of a patient so that a tether has a first end fixedly coupled to the prosthetic heart valve and a second free end, an intermediate portion of the tether extending through a wall of a heart of the patient while the prosthetic heart valve is in the native heart valve annulus;

sliding an epicardial anchor device over the intermediate portion of the tether so that the intermediate portion of the tether passes through a radial slot in a base of the epicardial anchor device and through a radial slot in a hub of the epicardial anchor device, until the intermediate portion of the tether is received within a central tether passageway of the epicardial anchor device, the radial slot of the base being aligned with the radial slot of the hub being continuous with the outer radial slot during the sliding;

positioning the epicardial anchor device in contact with the wall of the heart;

tensioning the tether to a desired tension level; and

while the tether is tensioned to the desired tension level, rotating the hub about a center longitudinal axis relative to the base to pin the tether to the epicardial anchor device; and/or

rotating the hub about a center longitudinal axis relative to the base causes a piercing portion of a locking pin assembly within the epicardial anchor device to drive through the central tether passageway and the intermediate portion of the tether.

According to still another aspect of the disclosure, a method of implanting a prosthetic heart valve system, the method comprises:

implanting a prosthetic heart valve into a native heart valve annulus of a patient so that a tether has a first end fixedly coupled to the prosthetic heart valve and a second free end, an intermediate portion of the tether extending through a wall of a heart of the patient while the prosthetic heart valve is in the native heart valve annulus;

sliding an epicardial anchor device over the intermediate portion of the tether so that the intermediate portion of the tether passes laterally through a radial slot of the epicardial anchor device, until the intermediate portion of the tether is received within a central tether passageway of the epicardial anchor device;

positioning the epicardial anchor device in contact with the wall of the heart;

manually actuating the epicardial anchor device to drive a locking pin through the central tether passageway and through the tether to lock the tether to the epicardial anchor device at a first tether tension;

engaging a tether tensioning tool with the epicardial anchor device while the tether is locked to the epicardial anchor device at the first tether tension;

fixing the tether to the tether tensioning tool while the tether tensioning tool is engaged with the epicardial anchor device;

after fixing the tether to the tether tensioning tool, actuating the epicardial anchor device using the tether tensioning tool to drive the locking pin away from the central tether passageway so that the locking pin disengages the tether;

while the locking pin is disengaged with the tether, using the tether tensioning tool to adjust the tether to a second tether tension different than the first tether tension; and

while the tether is at the second tether tension, using the tether tensioning tool to drive the locking pin through the central tether passageway and through the tether to lock the tether to the epicardial anchor device at the second tether tension; and/or

manually actuating the epicardial anchor device includes depressing a knob to drive the locking pin toward the central tether passageway; and/or

manually actuating the epicardial anchor device includes rotating an upper housing of the epicardial anchor device toward a lower housing of the epicardial anchor device, a pin within the upper housing pressing against an actuator at least partially within the lower housing as the upper housing rotates toward the lower housing.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above

Where schematics and/or embodiments described above indicate certain components arranged in certain orientations or positions, the arrangement of components may be modified. While the embodiments have been particularly shown and described, it will be understood that various changes in form and details may be made. Any portion of the apparatus and/or methods described herein may be combined in any combination, except mutually exclusive combinations. The embodiments described herein can include various combinations and/or sub-combinations of the functions, components, and/or features of the different embodiments described.

1. An epicardial anchor device comprising:
a base defining a radial slot; and
a hub defining a radial slot, the hub received at least partially within the base, the hub being rotatable about a center longitudinal axis of the hub relative to the base, the radial slot of the hub defining, at least in part, a tether passageway for receiving therethrough a tether coupled to a prosthetic heart valve,
wherein the epicardial anchor device has an unlocked configuration in which the radial slot of the base aligns with the radial slot of the hub so that the tether may be slid laterally through the radial slot of the base and the radial slot of the hub to position the tether at a radial center of the hub, and a locked configuration in which the radial slot of the base is out of alignment with the radial slot of the hub so that the tether cannot be slid laterally away from the radial center of the hub, the epicardial anchor device being transitionable from the locked configuration to the unlocked configuration via rotation of the hub about the center longitudinal axis of the hub relative to the base in a first rotational direction, and transitionable from the unlocked configuration to the locked configuration via rotation of the hub about the center longitudinal axis of the hub relative to the base in a second rotational direction opposite the first rotational direction.

2. The epicardial anchor device of claim 1, further comprising a locking pin assembly disposed within the epicardial anchor device.

3. The epicardial anchor device of claim 2, wherein a bottom surface of the hub includes a curved channel therein, the bottom surface of the hub confronting a top surface of the base.

4. The epicardial anchor device of claim 3, wherein a rod of the locking pin assembly is received within the curved channel.

5. The epicardial anchor device of claim 4, wherein a piercing portion of the locking pin assembly is configured to drive through the tether passageway upon transitioning the epicardial anchor device from the unlocked configuration to the locked configuration.

6. The epicardial anchor device of claim 1, further comprising a pad assembly coupled to a bottom surface of the base.

7. The epicardial anchor device of claim 6, wherein the pad assembly is formed of a fabric material, the pad assembly configured to contact a heart of the patient.

8. The epicardial anchor device of claim 1, wherein the radial slot of the base is an outer radial slot, and the radial slot of the hub is an inner radial slot.

9. The epicardial anchor device of claim 8, wherein in the unlocked configuration of the epicardial anchor device, the inner radial slot is continuous with the outer radial slot.

10. The epicardial anchor device of claim 9, wherein in the locked configuration of the epicardial anchor device, the inner radial slot is discontinuous with the outer radial slot.

11. The epicardial anchor device of claim 10, wherein the outer radial slot extends through an outer circumferential rim of the epicardial anchor device so that the outer circumferential rim is interrupted by the outer radial slot.

12. The epicardial anchor device of claim 1, further comprising a locking pin assembly, the locking pin assembly including a locking pin disposed within a locking pin channel within the base, and a rod extending transversely from the locking pin, the rod protruding through the base.

13. The epicardial anchor device of claim 12, wherein the hub includes an extension, a bottom surface of the extension defining a curved channel therein, the bottom surface of the extension confronting a top surface of the base.

14. The epicardial anchor device of claim 13, wherein the rod of the locking pin assembly is received within the curved channel of the extension.

15. A prosthetic heart valve system comprising;
the epicardial anchor device of claim 1;
the prosthetic heart valve; and
the tether, the tether having a first end coupled to the prosthetic heart valve and a second free end.

16. A method of implanting a prosthetic heart valve system, the method comprising:
implanting a prosthetic heart valve into a native heart valve annulus of a patient so that a tether has a first end fixedly coupled to the prosthetic heart valve and a second free end, an intermediate portion of the tether extending through a wall of a heart of the patient while the prosthetic heart valve is in the native heart valve annulus;
sliding an epicardial anchor device over the intermediate portion of the tether so that the intermediate portion of the tether passes through a radial slot in a base of the epicardial anchor device and through a radial slot in a hub of the epicardial anchor device, until the intermediate portion of the tether is received within a central tether passageway of the epicardial anchor device, the radial slot of the base being aligned with the radial slot of the hub being continuous with the outer radial slot during the sliding;
positioning the epicardial anchor device in contact with the wall of the heart;
tensioning the tether to a desired tension level; and
while the tether is tensioned to the desired tension level, rotating the hub about a center longitudinal axis relative to the base to pin the tether to the epicardial anchor device.

17. The method of claim 16, wherein rotating the hub about a center longitudinal axis relative to the base causes a piercing portion of a locking pin assembly within the epicardial anchor device to drive through the central tether passageway and the intermediate portion of the tether.

18. A method of implanting a prosthetic heart valve system, the method comprising:
implanting a prosthetic heart valve into a native heart valve annulus of a patient so that a tether has a first end fixedly coupled to the prosthetic heart valve and a second free end, an intermediate portion of the tether extending through a wall of a heart of the patient while the prosthetic heart valve is in the native heart valve annulus;
sliding an epicardial anchor device over the intermediate portion of the tether so that the intermediate portion of the tether passes laterally through a radial slot of the epicardial anchor device, until the intermediate portion of the tether is received within a central tether passageway of the epicardial anchor device;
positioning the epicardial anchor device in contact with the wall of the heart;
manually actuating the epicardial anchor device to drive a locking pin through the central tether passageway and through the tether to lock the tether to the epicardial anchor device at a first tether tension;
engaging a tether tensioning tool with the epicardial anchor device while the tether is locked to the epicardial anchor device at the first tether tension;
fixing the tether to the tether tensioning tool while the tether tensioning tool is engaged with the epicardial anchor device;
after fixing the tether to the tether tensioning tool, actuating the epicardial anchor device using the tether tensioning tool to drive the locking pin away from the central tether passageway so that the locking pin disengages the tether;
while the locking pin is disengaged with the tether, using the tether tensioning tool to adjust the tether to a second tether tension different than the first tether tension; and
while the tether is at the second tether tension, using the tether tensioning tool to drive the locking pin through the central tether passageway and through the tether to lock the tether to the epicardial anchor device at the second tether tension.

19. The method of claim 18, wherein manually actuating the epicardial anchor device includes depressing a knob to drive the locking pin toward the central tether passageway.

20. The method of claim 18, wherein manually actuating the epicardial anchor device includes rotating an upper housing of the epicardial anchor device toward a lower housing of the epicardial anchor device, a pin within the upper housing pressing against an actuator at least partially within the lower housing as the upper housing rotates toward the lower housing.
